(19) United States
(12) Patent Application Publication
(10) Pub. No.: US 2012/0164558 A1
(43) Pub. Date: Jun. 28, 2012

(54) BIPHENYLTETRASULFONIC ACID COMPOUND, METHOD FOR PRODUCING SAME, POLYMER AND POLYMER ELECTROLYTE

(75) Inventors: Noriyuki Hida, Sakai-shi (JP); Hiroaki Hibino, Toyonaka-shi (JP); Toru Onodera, Tsukuba-shi (JP)

(73) Assignee: Sumitomo Chemical Company, Limited, Chuo-ku, Tokyo (JP)

(21) Appl. No.: 13/394,232

(22) PCT Filed: Sep. 9, 2010
(86) PCT No.: PCT/JP2010/066001

§ 371 Date: Mar. 5, 2012

(30) Foreign Application Priority Data
Sep. 11, 2009 (JP) 2009-210147
Feb. 19, 2010 (JP) 2010-034430

(51) Int. Cl. H01M 8/10; B01J 31/10; C08G 75/24; C07C 309/39; C07C 303/22

(52) U.S. Cl. 429/494; 562/83; 521/25; 521/27; 502/159

(57) ABSTRACT

Provided are a biphenyltetrasulfonic acid compound represented by the formula (1):

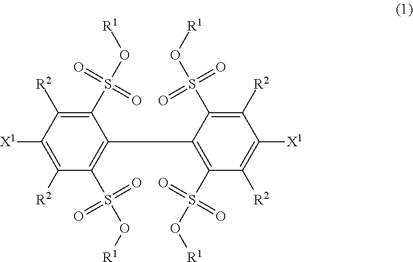

wherein R¹ represents a hydrogen atom, a cation, a hydrocarbon group, or the like; R² represents a hydrogen atom, an alkyl group, an aryl group, an aryloxy group, an aralkyl group, an aralkyoxy group, or the like; and X¹ represents a chlorine atom, a bromine atom, an iodine atom, a hydroxyl group, an amino group, or the like, and a polymer containing a structural unit originating from the biphenyltetrasulfonic acid compound.

### TECHNICAL FIELD

The present invention relates to a biphenyltetrasulfonic acid compound, a method for producing the same, a polymer, and a polymer electrolyte.

### BACKGROUND ART

As a monomer that imparts ion conductivity to a macromolecule having an elimination group such as aromatic polyether in which both ends have been chlorinated, a monomer having a partial structure of —SO₃— (hereinafter sometimes referred to as “a sulfonic acid group”) is known. As such a monomer having a sulfonic acid group, for example, di(2,2-dimethylpropyl) 4,4′-dichlorobiphenyl-2,2′-disulfonate, di(2,2-dimethylpropyl) 4,4′-dibromobiphenyl-2,2′-disulfonate, and diisopropyl 4,4′-dichlorobiphenyl-2,2′-disulfonate are known, and polymers having sulfonic acid groups obtained from these monomers are also known (see JP-2007-270118-A). In addition, it is known that the polymer having a sulfonic acid group is usable as a polymer electrolyte membrane for fuel cells (see JP-2007-177197-A).

### DISCLOSURE OF INVENTION

An object of the present invention is to provide a novel monomer that can impart ion conductivity to a macromolecule having an elimination group, a novel polymer that is obtained by polymerizing the monomer, a novel polymer electrolyte that contains the polymer, and the like.

Under these circumstances, the present inventors conducted intensive investigation regarding monomers having a sulfonic acid group, which resulted in the invention described below. That is, the present invention relates to:

<1> A biphenyltetrasulfonic acid compound represented by the formula (1):

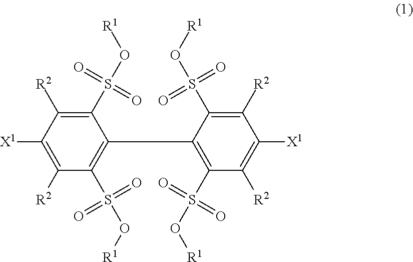

wherein R¹ each independently represents a hydrogen atom, a cation, or a hydrocarbon group having 1 to 20 carbon atoms that may have a substituent; R² each independently represents a hydrogen atom, an alkyl group having 1 to 20 carbon atoms that may have a substituent, an alkoxy group having 1 to 20 carbon atoms that may have a substituent, an aryl group having 6 to 20 carbon atoms that may have a substituent, an aryloxy group having 6 to 20 carbon atoms that may have a substituent, an aralkyl group having 7 to 20 carbon atoms that may have a substituent, or an aralkyloxy group having 7 to 20 carbon atoms that may have a substituent; and X¹ each independently represents a chlorine atom, a bromine atom, or an iodine atom;

<2> The biphenyltetrasulfonic acid compound according to <1>, wherein in the formula (1), at least one R¹ is a hydrogen atom or a cation, and at least one R² is a hydrogen atom;

<3> The biphenyltetrasulfonic acid compound according to <1> or <2>, wherein in the formula (1), at least one R¹ is an alkyl group having 1 to 6 carbon atoms;

<4> A method for producing a biphenyltetrasulfonic acid compound represented by the formula (1):

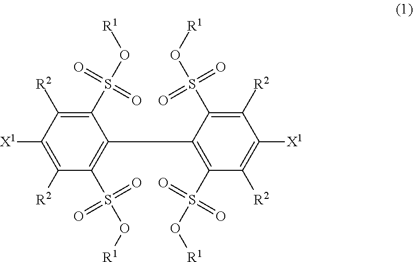

wherein R¹ each independently represents a hydrogen atom, a cation, or a hydrocarbon group having 1 to 20 carbon atoms that may have a substituent; R² each independently represents a hydrogen atom, an alkyl group having 1 to 20 carbon atoms that may have a substituent, an alkoxy group having 1 to 20 carbon atoms that may have a substituent, an aryl group having 6 to 20 carbon atoms that may have a substituent, an aryloxy group having 6 to 20 carbon atoms that may have a substituent, an aralkyl group having 7 to 20 carbon atoms that may have a substituent, or an aralkyloxy group having 7 to 20 carbon atoms that may have a substituent; X¹ represents a chlorine atom, a bromine atom, or an iodine atom; and X² represents a chlorine atom, a bromine atom, or an iodine atom), the method comprising:

a coupling reaction step of causing a coupling reaction of a benzenedisulfonic acid compound represented by the formula (2):

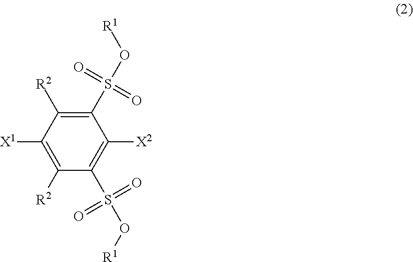

wherein R¹, R², and X¹ have the same definitions as those described above;

<5> The production method according to <4>, wherein the coupling reaction step is a step of causing a coupling reaction of the benzenedisulfonic acid compound represented by the formula (2) in the presence of metallic copper and monovalent copper halide;

<6> A method for producing a benzenedisulfonic acid compound represented by the formula (2)

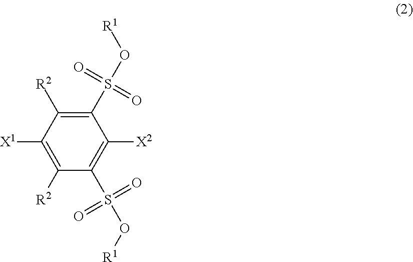

wherein R¹ each independently represents a hydrogen atom, a cation, or a hydrocarbon group having 1 to 20 carbon atoms that may have a substituent; R² each independently represents a hydrogen atom, an alkyl group having 1 to 20 carbon atoms that may have a substituent, an alkoxy group having 1 to 20 carbon atoms that may have a substituent, an aryl group having 6 to 20 carbon atoms that may have a substituent, an aryloxy group having 6 to 20 carbon atoms that may have a substituent, an aralkyl group having 7 to 20 carbon atoms that may have a substituent, or an aralkyloxy group having 7 to 20 carbon atoms that may have a substituent; X¹ represents a chlorine atom, a bromine atom, or an iodine atom; and X² represents a chlorine atom, a bromine atom, or an iodine atom, the method comprising:

a step of generating a diazonium compound by reacting an aniline compound represented by the formula (3):

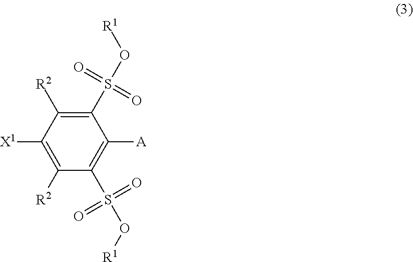

wherein R¹, R², and X¹ have the same definitions as those described above; and A represents NH₂, with a nitrous acid compound; and

a step of obtaining the benzenedisulfonic acid compound represented by the formula (2) by reacting the diazonium compound obtained in the above step with a halogen compound;

<7> A polymer comprising a structural unit originating from the biphenyltetrasulfonic acid compound according to any one of <I> to <3>;

<8> The polymer according to <7> further comprising a structural unit represented by the formula (X):

Ar⁰ (X)

wherein Ar⁰ represents an aromatic group which may have a substituent;

<9> The polymer according to <7> or <8> further comprising a structural unit represented by the formula (5):

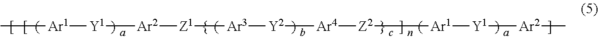

wherein a, b, and c each independently represents 0 or 1; n represents an integer of 2 or more; Ar¹, Ar², Ar³, and Ar⁴ each independently represents an aromatic group which may have a substituent; Y¹ and Y² each independently represents a single bond, a carbonyl group, a sulfonyl group, an isopropylidene group, a hexafluoroisopropylidene group, or a fluorene-9,9-diyl group; and Z¹ and Z² each independently represents an oxygen atom or a sulfur atom;

<10> The polymer according to <7> or <8> further comprising a structural unit represented by the formula (5′):

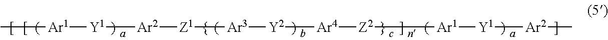

wherein a, b, and c each independently represents 0 or 1; n′ represents an integer of 5 or more; Ar¹, Ar², Ar³, and Ar⁴ each independently represents an aromatic group which may have a substituent; Y¹ and Y² each independently represents a single bond, a carbonyl group, a sulfonyl group, an isopropylidene group, a hexafluoroisopropylidene group, or a fluorene-9,9-diyl group; and Z¹ and Z² each independently represents an oxygen atom or a sulfur atom;

<11> The polymer according to <7> comprising a structural unit originating from the biphenyltetrasulfonic acid compound according to any one of <I> to <3>;

<12> A method for producing a polymer comprising a step of polymerizing a composition that contains a macromolecule including a structural unit represented by the formula (5):

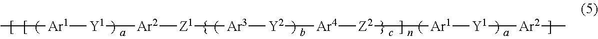

wherein a, b, and c each independently represents 0 or 1; n represents an integer of 2 or more; Ar¹, Ar², Ar³, and Ar⁴ each independently represents an aromatic group which may have a substituent; Y¹ and Y² each independently represents a single bond, a carbonyl group, a sulfonyl group, an isopropylidene group, a hexafluoroisopropylidene group, or a fluorene-9,9-diyl group; and Z¹ and Z² each independently represents an oxygen atom or a sulfur atom; and a biphenyltetrasulfonic acid compound represented by the formula (1):

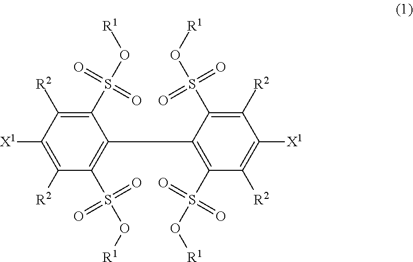

wherein R¹ each independently represents a hydrogen atom, a cation, or a hydrocarbon group having 1 to 20 carbon atoms that may have a substituent; R² each independently represents a hydrogen atom, an alkyl group having 1 to 20 carbon atoms that may have a substituent, an alkoxy group having 1 to 20 carbon atoms that may have a substituent, an aryl group having 6 to 20 carbon atoms that may have a substituent, an aryloxy group having 6 to 20 carbon atoms that may have a substituent, an aralkyl group having 7 to 20 carbon atoms that may have a substituent, or an aralkyloxy group having 7 to 20 carbon atoms that may have a substituent; and X¹ each independently represents a chlorine atom, a bromine atom, or an iodine atom, in the presence of a nickel compound;

<13> A method for producing a polymer comprising a step of polymerizing a composition that contains a macromolecule including a structural unit represented by the formula (5′):

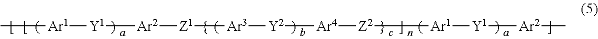

wherein a, b, and c each independently represents 0 or 1; n′ represents an integer of 5 or more; Ar¹, Ar², Ar^{a}, and Ar⁴ each independently represents an aromatic group which may have a substituent; Y¹ and Y² each independently represents a single bond, a carbonyl group, a sulfonyl group, an isopropylidene group, a hexafluoroisopropylidene group, or a fluorene-9,9-diyl group; and Z¹ and Z² each independently represents an oxygen atom or a sulfur atom; and a biphenyltetrasulfonic acid compound represented by the formula (1):

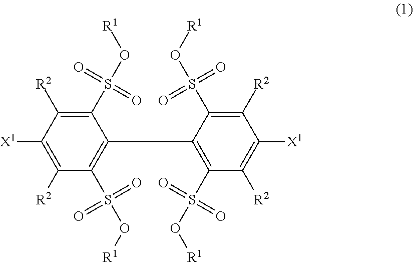

wherein R¹ each independently represents a hydrogen atom, a cation, or a hydrocarbon group having 1 to 20 carbon atoms that may have a substituent; R² each independently represents a hydrogen atom, an alkyl group having 1 to 20 carbon atoms that may have a substituent, an alkoxy group having 1 to 20 carbon atoms that may have a substituent, an aryl group having 6 to 20 carbon atoms that may have a substituent, an aryloxy group having 6 to 20 carbon atoms that may have a substituent, an aralkyl group having 7 to 20 carbon atoms that may have a substituent, or an aralkyloxy group having 7 to 20 carbon atoms that may have a substituent; and X¹ each independently represents a chlorine atom, a bromine atom, or an iodine atom, in the presence of a nickel compound; or the like. In addition, the present invention also includes the following inventions:

<14> A polymer electrolyte comprising the polymer according to any one of <7> to <11>;

<15> A polymer electrolyte membrane comprising the polymer electrolyte according to <14>;

<16> A polymer electrolyte composite membrane comprising the polymer electrolyte according to <14> and a porous substrate;

<17> A catalyst composition comprising the polymer electrolyte according to <14> and a catalyst component;

<18> A membrane electrode assembly comprising at least one kind selected from the group consisting of the polymer electrolyte membrane according to <15>, the polymer electrolyte composite membrane according to <16>, and the catalyst composition according to <17>; and

<19> A polymer electrolyte fuel cell comprising the membrane electrode assembly according to <18>.

According to the present invention, it is possible to provide a monomer that can impart ion conductivity to a macromolecule having an elimination group, a novel polymer that is obtained by polymerizing the monomer, a novel polymer electrolyte that contains the polymer, and the like.

### EMBODIMENT FOR CARRYING OUT THE INVENTION

Hereinafter, the present invention will be described in detail.

The present invention relates to a biphenyltetrasulfonic acid compound represented by the formula (1).

In the formula (1), R¹ each independently represents a hydrogen atom, a cation, or a hydrocarbon group having 1 to 20 carbon atoms that may have a substituent.

When R¹ is a cation, this R¹ and an oxygen atom contained in the partial structure of —SO₃— (sulfonic acid group) are combined together by an ionic bond. Specifically, for example, when the cation is a sodium ion (Na⁺), —SO₃—Na⁺ is formed.

Herein, examples of the cation include alkali metal ions such as a lithium ion (Li⁺), a sodium ion (Na⁺), a potassium ion (K⁺), and a cesium ion (Cs⁺); and ammonium ions such as an ammonium ion (NH₄⁺), a methylammonium ion (CH₃NH₃⁺), a diethylammonium ion, a tri(n-propyl)ammonium ion, a tetra(n-butyl)ammonium ion, a diisopropyl diethylammonium ion, a tetra(n-octyl)ammonium ion, a tetra(n-decyl)ammonium ion, and a triphenylammonium ion.

When R¹ is a hydrogen atom or the hydrocarbon group described above, this R¹ and an oxygen atom contained in the sulfonic acid group are combined together by a covalent bond. Specifically, for example, when the hydrocarbon group is a methyl group (Me), —SO₃Me is formed.

Examples of the hydrocarbon group having 1 to 20 carbon atoms that may have a substituent include a linear, branched, or cyclic alkyl group such as a methyl group, an ethyl group, an n-propyl group, an isopropyl group, an n-butyl group, an isobutyl group, a sec-butyl group, a tert-butyl group, an n-pentyl group, a 2,2-dimethyl-1-propyl group, a cyclopentyl group, an n-hexyl group, a cyclohexyl group, an n-heptyl group, a 2-methylpentyl group, an n-octyl group, a 2-ethylhexyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, or an n-eicosyl group; and

an aryl group such as a phenyl group, a 2-tolyl group, a 3-tolyl group, a 4-tolyl group, a 2,3-xylyl group, a 2,4-xylyl group, a 2,5-xylyl group, a 2,6-xylyl group, a 3,4-xylyl group, a 3,5-xylyl group, a 2,3,4-trimethylphenyl group, a 2,3,5-trimethylphenyl group, a 2,3,6-trimethylphenyl group, a 2,4,6-trimethylphenyl group, a 3,4,5-trimethylphenyl group, a 2,3,4,5-tetramethylphenyl group, a 2,3,4,6-tetramethylphenyl group, a 2,3,5,6-tetramethylphenyl group, a pentamethylphenyl group, an ethylphenyl group, an n-propylphenyl group, an isopropylphenyl group, an n-butylphenyl group, a sec-butylphenyl group, a tert-butylphenyl group, an n-pentylphenyl group, a neopentylphenyl group, an n-hexylphenyl group, an n-octylphenyl group, an n-decylphenyl group, an n-dodecylphenyl group, an n-tetradecylphenyl group, a naphthyl group, or an anthracenyl group.

Examples of the substituent that the hydrocarbon group may have include a fluorine atom; a cyano group; a linear, branched, or cyclic alkoxy group having 1 to 20 carbon atoms such as a methoxy group, an ethoxy group, an n-propoxy group, an isopropoxy group, an n-butoxy group, an isobutoxy group, a sec-butoxy group, a tert-butoxy group, an n-pentyloxy group, a 2,2-dimethyl-1-propoxy group, a cyclopentyloxy group, an n-hexyloxy group, a cyclohexyloxy group, an n-heptyloxy group, a 2-methylpentyloxy group, an n-octyloxy group, a 2-ethylhexyloxy group, an n-nonyloxy group, an n-decyloxy group, an n-undecyloxy group, an n-dodecyloxy group, an n-tridecyloxy group, an n-tetradecyloxy group, an n-pentadecyloxy group, an n-hexadecyloxy group, an n-heptadecyloxy group, an n-octadecyloxy group, an n-nonadecyloxy group, or an n-eicosyloxy group; the aryl group exemplified as above; and

an aryloxy group having 6 to 20 carbon atoms that includes the aryl group exemplified as above and an oxygen atom.

Examples of preferable R¹s include a hydrogen atom, an alkali metal ion, and an alkyl group having 1 to 20 carbon atoms that may have a substituent. Examples of more preferable R¹s include a hydrogen atom, a sodium ion (Na⁺), a 2,2-dimethylpropyl group, and a diisopropyl group.

When the biphenyltetrasulfonic acid compound of the present invention is used as a monomer imparting ion conductivity, as R¹, at least two R¹s in a molecule, and preferably three or four R¹s in a molecule are hydrocarbon groups that can be deprotected with an acid, a base, or a halogen compound. That is, R¹ is a hydrocarbon group that can be deprotected as R¹OH from —OR¹ in the formula (1). As such a hydrocarbon group, for example, a 2,2-dimethylpropyl group and a diisopropyl group are preferable.

In the formula (1), R² each independently represents a hydrogen atom, an alkyl group having 1 to 20 carbon atoms that may have a substituent, an alkoxy group having 1 to 20 carbon atoms that may have a substituent, an aryl group having 6 to 20 carbon atoms that may have a substituent, an aryloxy group having 6 to 20 carbon atoms that may have a substituent, an aralkyl group having 7 to 20 carbon atoms that may have a substituent, or an aralkyloxy group having 7 to 20 carbon atoms that may have a substituent.

Herein, examples of the alkyl group having 1 to 20 carbon atoms that may have a substituent, the alkoxy group having 1 to 20 carbon atoms that may have a substituent, the aryl group having 6 to 20 carbon atoms that may have a substituent, and the aryloxy group having 6 to 20 carbon atoms that may have a substituent include those exemplified above as R¹.

Examples of the aralkyl group having 7 to 20 carbon atoms include a benzyl group, a (2-methylphenyl)methyl group, a (3-methylphenyl)methyl group, a (4-methylphenyl)methyl group, a (2,3-dimethylphenyl)methyl group, a (2,4-dimethylphenyl)methyl group, a (2,5-dimethylphenyl)methyl group, a (2,6-dimethylphenyl)methyl group, a (3,4-dimethylphenyl)methyl group, a (4,6-dimethylphenyl)methyl group, a (2,3,4-trimethylphenyl)methyl group, a (2,3,5-trimethylphenyl)methyl group, a (2,3,6-trimethylphenyl)methyl group, a (3,4,5-trimethylphenyl)methyl group, a (2,4,6-trimethylphenyl)methyl group, a (2,3,4,5-tetramethylphenyl)methyl group, a (2,3,4,6-tetramethylphenyl)methyl group, a (2,3,5,6-tetramethylphenyl)methyl group, a (pentamethylphenyl)methyl group, an (ethylphenyl)methyl group, an (n-propylphenyl)methyl group, an (isopropylphenyl)methyl group, an (n-butylphenyl)methyl group, a (sec-butylphenyl)methyl group, a (tert-butylphenyl)methyl group, an (n-pentylphenyl)methyl group, a (neopentylphenyl)methyl group, an (n-hexylphenyl)methyl group, an (n-octylphenyl)methyl group, an (n-decylphenyl)methyl group, an (n-decylphenyl)methyl group, a naphthylmethyl group, and an anthracenylmethyl group.

Examples of the substituent that the aralkyl group may have include the substituents exemplified as above.

Examples of the aralkyloxy group having 7 to 20 carbon atoms include a benzyloxy group, a (2-methylphenyl)methoxy group, a (3-methylphenyl)methoxy group, a (4-methylphenyl)methoxy group, a (2,3-dimethylphenyl)methoxy group, a (2,4-dimethylphenyl)methoxy group, a (2,5-dimethylphenyl)methoxy group, a (2,6-dimethylphenyl)methoxy group, a (3,4-dimethylphenyl)methoxy group, a (3,5-dimethylphenyl)methoxy group, a (2,3,4-trimethylphenyl)methoxy group, a (2,3,5-trimethylphenyl)methoxy group, a (2,3,6-trimethylphenyl)methoxy group, a (2,4,5-trimethylphenyl)methoxy group, a (2,4,6-trimethylphenyl)methoxy group, a (3,4,5-trimethylphenyl)methoxy group, a (2,3,4,5-tetramethylphenyl)methoxy group, a (2,3,4,6-tetramethylphenyl)methoxy group, a (2,3,5,6-tetramethylphenyl)methoxy group, a (pentamethylphenyl)methoxy group, an (ethylphenyl)methoxy group, an (n-propylphenyl)methoxy group, an (isopropylphenyl)methoxy group, an (n-butylphenyl)methoxy group, a (sec-butylphenyl)methoxy group, a (tert-butylphenyl)methoxy group, an (n-hexylphenyl)methoxy group, an (n-octylphenyl)methoxy group, an (n-decylphenyl)methoxy group, a naphthylmethoxy group, and an anthracenylmethoxy group.

Examples of the substituent that the aralkyloxy group described above may have include the substituents exemplified as above.

R²s in one molecule of the biphenyltetrasulfonic acid compound represented by the formula (1) may be the same as or different from each other. However, for the ease of the production in the method for producing the biphenyltetrasulfonic acid compound described later, all R²s are preferably the same as each other.

Examples of preferable R²s include a hydrogen atom and an alkyl group having 1 to 20 carbon atoms, and examples of more preferable R²s include a hydrogen atom. In addition, at least one of four R²s in a molecule is preferably a hydrogen atom, but for the ease of production, a biphenyltetrasulfonic acid compound in which at least two or more out of four R²s in a molecule are hydrogen atoms is more preferable, and a biphenyltetrasulfonic acid compound in which all of four R²s in a molecule are hydrogen atoms is even more preferable.

In the formula (1), X¹ each independently represents a chlorine atom, a bromine atom, or an iodine atom.

X¹s in a molecule may be the same as or different from each other, but for the ease of production, a compound in which all X¹s in a molecule are the same as each other is preferable.

Examples of preferable X¹s include a chlorine atom and a bromine atom, and examples of more preferable X¹ s include a chlorine atom.

Examples of the biphenyltetrasulfonic acid compound represented by the formula (1) include 4,4′-dichloro-2,2′,6,6′-biphenyltetrasulfonic acid, tetrasodium 4,4′-dichloro-2,2′,6,6′-biphenyltetrasulfonate, tetrasodium 4,4′-dibromo-2,2′,6,6′-biphenyltetrasulfonate, tetrasodium 4,4′-diiodo-2,2′,6,6′-biphenyltetrasulfonate, tetramethyl 4,4′-dichloro-2,2′,6,6′-biphenyltetrasulfonate, tetraethyl 4,4′-dichloro-2,2′,6,6′-biphenyltetrasulfonate, tetrakis(2,2-dimethyl-1-propyl) 4,4′-dichloro-2,2′,6,6′-biphenyltetrasulfonate, tetraphenyl 4,4′-dichloro-2,2′,6,6′-biphenyltetrasulfonate, tetraammoniurn 4,4′-dichloro-2,2′,6,6′-biphenyltetrasulfonate, dimethyl disodium 4,4′-dichloro-2,2′,6,6′-biphenyltetrasulfonate, and tris(2,2-dimethyl-1-propyl) sodium 4,4′-dichloro-2,2′,6,6′-biphenyltetrasulfonate.

Another example of the biphenyltetrasulfonic acid compound represented by the formula (1) includes a compound in which R¹ is a hydrocarbon group having 1 to 20 carbon atoms that may have a substituent. A more preferable example includes a biphenyltetrasulfonic acid compound in which R¹ is an alkyl group having 1 to 6 carbon atoms, R² is a hydrogen atom, and X¹ is a chlorine atom, a bromine atom, or an iodine atom.

Specific examples of the biphenyltetrasulfonic acid compound include tetramethyl 4,4′-dichloro-2,2′,6,6′-biphenyltetrasulfonate, tetraethyl 4,4′-dichloro-2,2′,6,6′-biphenyltetrasulfonate, tetrakis(2,2-dimethyl-1-propyl) 4,4′-dichloro-2,2′,6,6′-biphenyltetrasulfonate, and tetraphenyl 4,4′-dichloro-2,2′,6,6′-biphenyltetrasulfonate.

When the biphenyltetrasulfonic acid compound represented by the formula (1) is used as a monomer imparting ion conductivity to a polymer, for the ease of producing the polymer containing the compound, the compound is preferably a biphenyltetrasulfonic acid compound in which at least two R¹s in a molecule are hydrocarbon groups having 1 to 20 carbon atoms that may have a substituent. Examples of the method for producing the biphenyltetrasulfonic acid compound include a method of protecting the biphenyltetrasulfonic acid compound in which all R¹s in the formula (1) are cations with alcohol.

Specifically, examples of such a method include a method comprising [1] reacting the biphenyltetrasulfonic acid compound represented by the formula (1) in which R¹ is a cation with a sulfurous acid halide such as thionyl chloride in the presence of an organic base such as N,N-dimethylformamide, [2] separately preparing an alkoxide by reacting an alcohol with a base such as butyllithium, and [3] mixing a mass obtained from the reaction of [1] with a mass obtained from the reaction of [2].

Regarding the biphenyltetrasulfonic acid compound represented by the formula (1), examples of a production method different from the above method include a method comprising a step of causing a coupling reaction (hereinafter sometimes referred to as a coupling reaction step) of a benzenedisulfonic acid compound represented by the formula (2):

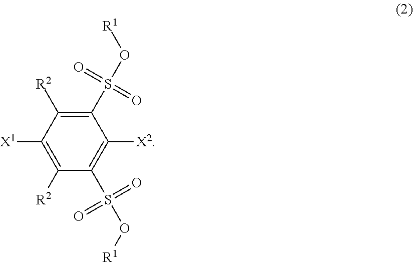

Herein, X² represents a chlorine atom, a bromine atom, or an iodine atom, and preferably represents a bromine atom or an iodine atom. More preferably, when X¹ is a chlorine atom, X² is preferably a bromine atom or an iodine atom, and when X¹ is a bromine atom, X² is preferably an iodine atom.

The coupling reaction step is preferably performed in the presence of, for example, a single transition metal and/or a transition metal compound. When a single transition metal and a transition metal compound are used concurrently, the single transition metal and the respective transition metal elements in the transition metal compound may be the same as or different from each other.

Examples of the transition metal elements include copper.

When copper is used as a single transition metal in the coupling reaction step, metallic copper is preferable. The amount of the metallic copper used is, for example, in a range of from 0.5 mol to 10 mol based on 1 mol of the benzenedisulfonic acid compound represented by the formula (2). If the amount is 0.5 mol or more, the post-treatment tends to be easier, and if the amount is 10 mol or less, the yield tends to be increased.

The form of the metallic copper can be, for example, powder, flakes, or particles, and in terms of operability, a powder form is preferable. Such a metallic copper is easily commercially available.

In the commercially available metallic copper, only a small portion of the surface thereof turns into copper oxide due to oxidation caused by oxygen in the environment in some cases. The metallic copper including copper oxide may be provided as it is to the coupling reaction step, or may be provided to the coupling reaction step after the copper oxide is removed.

When the metallic copper is used in the coupling reaction step, it is preferable to concurrently use monovalent copper halide as the transition metal compound. Examples of the monovalent copper halide include copper chloride, copper bromide, and copper iodide, and among these, copper iodide is preferable. The amount of the monovalent copper halide used is, for example, in a range of from 0.1 mol to 50 mol, and preferably is in a range of from 0.5 mol to 10 mol, based on 1 mol of the benzenedisulfonic acid compound represented by the formula (2).

The coupling reaction step is preferably performed in the presence of a solvent. The solvent may be a solvent that can dissolve the biphenyltetrasulfonic acid compound represented by the formula (1) and the benzenedisulfonic acid compound represented by the formula (2). Specific examples of the solvent include an aromatic hydrocarbon solvent such as toluene or xylene; an ether solvent such as tetrahydrofuran, 1,4-dioxane, or diethylene glycol dimethyl ether; an aprotonic polar solvent such as dimethyl sulfoxide, N-methyl-2-pyrrolidone, N,N-dimethylformamide, N,N-dimethylacetamide, or hexamethylphosphoric triamide; and a halogenated hydrocarbon solvent such as dichloromethane or dichloroethane. These solvents may be used alone or as a mixture of two or more kinds thereof.

Examples of a preferable solvent include an aprotonic polar solvent, and examples of a more preferable solvent include N-methyl-2-pyrrolidone and N,N-dimethylformamide.

The amount of the solvent used is, for example, in a range of from 0.5 parts by weight to 20 parts by weight, and preferably is in a range of from 1 parts by weight to 10 parts by weight, based on 1 part by weight of the benzenedisulfonic acid compound represented by the formula (2).

The coupling reaction step is preferably performed, for example, in an atmosphere of inert gas such as nitrogen gas.

The reaction temperature in the coupling reaction step is in a range of, for example, from 0° C. to 300° C., preferably in a range of, for example, from 50° C. to 250° C., more preferably in a range of for example, from 100° C. to 200° C., and even more preferably in a range of, for example, from 140° C. to 180° C. If the reaction temperature is 0° C. or higher, the yield of the biphenyltetrasulfonic acid compound represented by the formula (1) tends to be increased, and if the reaction temperature is 300° C. or lower, a side reaction such as a degradation reaction tends to be inhibited.

The reaction time in the coupling reaction step is in a range of, for example, from 1 hour to 48 hours.

Examples of the method for producing the benzenedisulfonic acid compound represented by the formula (2), which is provided to the coupling reaction step, include a method of producing by a reaction (so-called Sandmeyer reaction) that comprises a step of generating a diazonium compound by reacting a compound represented by the formula (3):

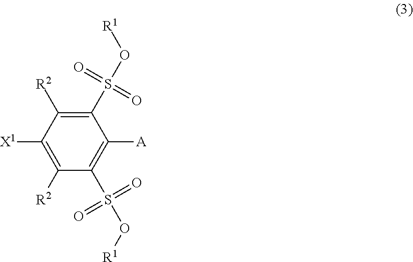

wherein R¹, R², and X¹ have the same definitions as those described above; and A represents NH₂ (hereinafter sometimes referred to as an aniline compound), with a nitrous acid compound, and a step of obtaining the benzenedisulfonic acid compound represented by the formula (2) by means of reacting the diazonium compound obtained in the above step with a halogen compound.

Examples of the nitrous acid compound include a nitrous acid alkali metal salt such as sodium nitrite or potassium nitrite, and nitrous acid alkyl ester having an alkyl group with 1 to 6 carbon atoms such as ethyl nitrite or t-butyl nitrite. The amount of the nitrous acid compound used is in a range of, for example, from 0.8 mol to 1.5 mol based on 1 mol of the aniline compound. Such a nitrous acid compound may be used without being diluted or used as a solution by being dissolved in water or the like.

Examples of the method of reacting the aniline compound with the nitrous acid compound include a method of adding the nitrous acid compound to an acidic solution containing the aniline compound. The temperature at the time of adding the nitrous acid compound is in a range of, for example, from −30° C. to 40° C., and the temperature is preferably in a range of, for example, from −10° C. to 20° C.

By performing the step of reacting the nitrous acid compound, a diazonium compound in which A in the aniline compound represented by the formula (3) has been substituted with a diazonio group (—N⁺≡N) is obtained.

After the step of obtaining the diazonium compound described above, a step of obtaining the benzenedisulfonic acid compound represented by the formula (2) by means of reacting the diazonium compound obtained in the above step with a halogen compound is performed. Examples of the halogen compound used in this step include monovalent copper halides such as copper (I) chloride, copper (I) bromide, copper (I) oxide, copper (I) iodide, or copper (I) cyanide; divalent copper halides such as copper (II) chloride, copper (II) bromide, copper (II) oxide, copper (II) iodide, copper (II) cyanide, copper (II) sulfate, or copper (II) acetate; alkali metal halides such as sodium iodide, potassium bromide, or potassium iodide; and hydrogen halides such as hydrogen chloride, hydrogen bromide, or hydrogen iodide. These halogen compounds may be used alone or in combination of two or more kinds thereof.

It is preferable to use two or more kinds of halogen compounds in combination. Examples of the combination include a combination of a monovalent copper halide and a hydrogen halide such as a combination of copper (I) chloride and hydrogen chloride, a combination of copper (I) bromide and hydrogen bromide, a combination of copper (I) chloride and hydrogen iodide, a combination of copper (I) bromide and hydrogen chloride, a combination of copper (I) bromide and hydrogen bromide, a combination of copper (I) bromide and hydrogen iodide, a combination of copper (I) iodide and hydrogen chloride, a combination of copper (I) iodide and hydrogen iodide, or a combination of copper (I) iodide and hydrogen iodide; and a combination of a monovalent copper halide, a hydrogen halide, and a metal halide such as a combination of copper (I) bromide, hydrogen bromide, and potassium bromide, a combination of copper (I) bromide, hydrogen bromide, and potassium iodide, a combination of copper (I) bromide, hydrogen iodide, and potassium bromide, a combination of copper (I) bromide, hydrogen iodide, and potassium iodide, a combination of copper (I) iodide, hydrogen iodide, and potassium iodide, or a combination of copper (1) chloride, hydrogen iodide, and potassium iodide.

The amount of the halogen compound used is in a range of, for example, from 0.5 mol to 10 mol, and preferably is in a range of, for example, from 1 mol to 5 mol, based on 1 mol of the diazonium compound.

The reaction temperature in the step of obtaining the benzenedisulfonic acid compound represented by the formula (2) is, for example, in a range of from −10° C. to 100° C., and preferably is in a range of from 0° C. to 70° C.

The aniline compound represented by the formula (3) can be prepared by, for example, a method of sulfonating a compound represented by the formula (4):

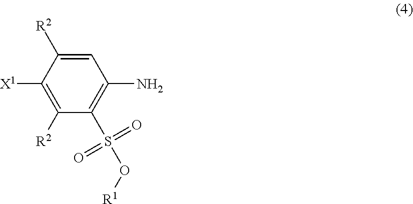

wherein R¹, R², and X¹ have the same definitions as those described above, with sulfuric acid and/or fuming sulfuric acid (see Collection of Czechoslovak Chemical Communications, 1964, 29, (1969)).

The polymer of the present invention is a polymer having a structural unit originating from the biphenyltetrasulfonic acid compound represented by the formula (1), and the polymer is usable as a polymer electrolyte since the polymer has ion conductivity. As the structural unit originating from the biphenyltetrasulfonic acid compound represented by the formula (1), for example, a structural unit represented by the formula (1′):

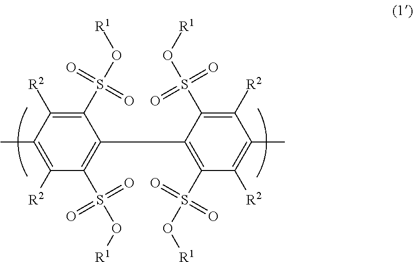

in the formula (1′), R¹ and R² have the same definitions as those described above, is preferable.

Examples of the polymer of the present invention include a homopolymer of the biphenyltetrasulfonic acid compound represented by the formula (1), a copolymer of the biphenyltetrasulfonic acid compound represented by the formula (1) and another monomer, and a copolymer of aromatic polyether and the biphenyltetrasulfonic acid compound represented by the formula (1).

Herein, the aromatic polyether refers to a macromolecule having a structural unit comprising an aromatic group which may have a substituent and an ether bond, and the ether bond refers to —O— (ether bond) or —S-(thioether bond).

The polymer is preferably water-insoluble. The term “water-insoluble” means that solubility in water at 23° C. is 5% by weight or less. Such a water-insoluble polymer can be prepared by copolymerizing the biphenyltetrasulfonic acid compound represented by the formula (1) with another monomer.

Examples of a preferable copolymer include a polymer which has a structural unit represented by the formula (X) and a structural unit originating from the biphenyltetrasulfonic acid compound represented by the formula (1).

Ar⁰ (X)

In the formula (X), Ar⁰ represents an aromatic group. Examples of the aromatic group include a monocyclic aromatic group such as 1,3-phenylene or 1,4-phenylene; a ring-condensed aromatic group such as 1,3-naphthalenediyl, 1,4-naphthalenediyl, 1,5-naphthalenediyl, 1,6-naphthalenediyl, 1,7-naphthalenediyl, 2,6-naphthalenediyl, or 2,7-naphthalenediyl; and a hetero aromatic group such as pyridinediyl, quinoxalinediyl, or thiophenediyl. Among these, a monocyclic aromatic group is preferable.

To the aromatic group represented by Ar⁰, a fluorine atom, an alkyl group, an alkoxy group, an aryl group, an aryloxy group, or an acyl group may be bound, and these groups may further have a substituent.

Examples of the alkyl group which may have a substituent include an alkyl group having 1 to 10 carbon atoms such as methyl, ethyl, n-propyl, isopropyl, n-butyl, sec-butyl, isobutyl, n-pentyl, 2,2-dimethylpropyl, cyclopentyl, n-hexyl, cyclohexyl, 2-methylpentyl, 2-ethyhexyl, or nonyl; and an alkyl group in which a substituent such as a fluorine atom, a hydroxyl group, a cyano group, an amino group, a methoxy group, an ethoxy group, an isopropyloxy group, a phenyl group, a naphthyl group, a phenoxy group, or a naphthyloxy group binds to the above alkyl groups.

Examples of the alkoxy group which may have a substituent include an alkoxy group having 1 to 10 carbon atoms such as methoxy, ethoxy, n-propyloxy, isopropyloxy, n-butyloxy, sec-butyloxy, tert-butyloxy, isobutyloxy, n-pentyloxy, 2,2-dimethylpropyloxy, cyclopentyloxy, n-hexyloxy, cyclohexyloxy, 2-methylpentyloxy, or 2-ethylhexyloxy; and an alkoxy group in which a substituent such as a fluorine atom, a hydroxyl group, a cyano group, an amino group, a methoxy group, an ethoxy group, an isopropyloxy group, a phenyl group, a naphthyl group, a phenoxy group, or a naphthyloxy group binds to the above alkoxy groups.

Examples of the aryl group which may have a substituent include an aryl group having 6 to 10 carbon atoms such as phenyl or naphthyl; and an aryl group in which a substituent such as a fluorine atom, a hydroxyl group, a cyano group, an amino group, a methoxy group, an ethoxy group, an isopropyloxy group, a phenyl group, a naphthyl group, a phenoxy group, or a naphthyloxy group binds to the above aryl groups.

Examples of the aryloxy group which may have a substituent include an aryloxy group having 6 to 10 carbon atoms such as phenoxy or naphthyloxy; and an aryloxy group in which a substituent such as a fluorine atom, a hydroxyl group, a cyano group, an amino group, a methoxy group, an ethoxy group, an isopropyloxy group, a phenyl group, a naphthyl group, a phenoxy group, or a naphthyloxy group binds to the above aryloxy groups.

Examples of the acyl group which may have a substituent include an acyl group having 2 to 20 carbon atoms such as acetyl, propionyl, butyryl, isobutyryl, benzoyl, 1-naphthoyl, or 2-naphthoyl; and an acyl group in which a substituent such as a fluorine atom, a hydroxyl group, a cyano group, an amino group, a methoxy group, an ethoxy group, an isopropyloxy group, a phenyl group, a naphthyl group, a phenoxy group, or a naphthyloxy group binds to the above acyl groups.

When the aromatic group represented by Ar⁰ includes an acyl group which may have a substituent, two structural units having the acyl group are adjacent to each other, and the acyl groups of the two structural units bind to each other; alternatively, after the acyl groups bind to each other in this manner, a rearrangement reaction is caused, in some cases. Whether or not the reaction in which substituents on the aromatic ring bind to each other or the rearrangement reaction is caused after the substituents bind to each other can be confirmed by, for example, measuring a ¹³C-nuclear magnetic resonance spectrum.

Examples of the compound having the structural unit represented by the formula (X) include a compound (hereinafter, abbreviated to a compound (Y)) which has a group in the structural unit represented by the formula (X) that is able to form a bond by reacting with X¹ of the biphenyltetrasulfonic acid compound represented by the formula (1), and which has a plurality of elimination groups such as halogen atoms.

Examples of a preferable copolymer include a polymer containing a structural unit represented by the formula (5):

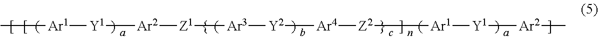

wherein a, b, and c each independently represents 0 or 1; n represents an integer of 2 or more; Ar¹, Ar², Ar³, and Ar⁴ each independently represents an aromatic group; herein, the aromatic group may have one or more substituents selected from the group consisting of an alkyl group having 1 to 20 carbon atoms that may have one or more substituents selected from the group consisting of a fluorine atom, a cyano group, an alkoxy group having 1 to 20 carbon atoms, an aryl group having 6 to 20 carbon atoms, and an aryloxy group having 6 to 20 carbon atoms; an alkoxy group having 1 to 20 carbon atoms that may have one or more substituents selected from the group consisting of a fluorine atom, a cyano group, an alkoxy group having 1 to 20 carbon atoms, an aryl group having 6 to 20 carbon atoms, and an aryloxy group having 6 to 20 carbon atoms; an aryl group having 6 to 20 carbon atoms that may have one or more substituents selected from the group consisting of a fluorine atom, a cyano group, an alkoxy group having 1 to 20 carbon atoms, and an aryloxy group having 6 to 10 carbon atoms; an aryloxy group having 6 to 20 carbon atoms that may have one or more substituents selected from the group consisting of a fluorine atom, a cyano group, an alkoxy group having 1 to 20 carbon atoms, and an aryloxy group having 6 to 20 carbon atoms; and an acyl group having 2 to 20 carbon atoms that may have one or more substituents selected from the group consisting of a fluorine atom, a cyano group, an alkoxy group having 1 to 20 carbon atoms, an aryl group having 6 to 20 carbon atoms, and an aryloxy group having 6 to 20 carbon atoms; Y¹ and Y² each independently represents a single bond, a carbonyl group, a sulfonyl group, an isopropylidene group, a hexafluoroisopropylidene group, or a fuorene-9,9-diyl group; and Z¹ and Z² each independently represents an oxygen atom or a sulfur atom, and

a structural unit originating from the biphenyltetrasulfonic acid compound represented by the formula (1).

a, b, and c each independently represents 0 or 1. n represents an integer of 2 or more, preferably an integer in a range of, for example, from 2 to 200, and more preferably an integer in a range of, for example, from 5 to 200.

Ar¹, Ar², Ar³, and Ar⁴ each independently represents an aromatic group. Examples of the aromatic group include a monocyclic aromatic group such as 1,3-phenylene or 1,4-phenylene; a ring-condensed aromatic group such as 1,3-naphthalenediyl, 1,4-naphthalenediyl, 1,5-naphthalenediyl, 1,6-naphthalenediyl, 1,7-naphthalenediyl, 2,6-naphthalenediyl, or 2,7-naphthalenediyl; and a hetero aromatic group such as pyridinediyl, quinoxalinediyl, or thiophenediyl. Among these, a monocyclic aromatic group is preferable.

In addition, to the aromatic group represented by Ar¹, Ar², Ar³, or Ar⁴, a fluorine atom, an alkyl group, an alkoxy group, an aryl group, an aryloxy group, or an acyl group may be bound, and these groups may further have a substituent.

Examples of the alkyl group which may have a substituent include an alkyl group having 1 to 10 carbon atoms such as methyl, ethyl, n-propyl, isopropyl, n-butyl, sec-butyl, isobutyl, n-pentyl, 2,2-dimethylpropyl, cyclopentyl, n-hexyl, cyclohexyl, 2-methylpentyl, 2-ethyhexyl, or nonyl; and an alkyl group in which a substituent such as a fluorine atom, a hydroxyl group, a cyano group, an amino group, a methoxy group, an ethoxy group, an isopropyloxy group, a phenyl group, a naphthyl group, a phenoxy group, or a naphthyloxy group binds to the above alkyl groups.

Examples of the alkoxy group which may have a substituent include an alkoxy group having 1 to 10 carbon atoms such as methoxy, ethoxy, n-propyloxy, isopropyloxy, n-butyloxy, sec-butyloxy, tert-butyloxy, isobutyloxy, n-pentyloxy, 2,2-dimethylpropyloxy, cyclopentyloxy, n-hexyloxy, cyclohexyloxy, 2-methylpentyloxy, or 2-ethylhexyloxy; and an alkoxy group in which a substituent such as a fluorine atom, a hydroxyl group, a cyano group, an amino group, a methoxy group, an ethoxy group, an isopropyloxy group, a phenyl group, a naphthyl group, a phenoxy group, or a naphthyloxy group binds to the above alkoxy groups.

Examples of the aryl group which may have a substituent include an aryl group having 6 to 10 carbon atoms such as phenyl or naphthyl; and an aryl group in which a substituent such as a fluorine atom, a hydroxyl group, a cyano group, an amino group, a methoxy group, an ethoxy group, an isopropyloxy group, a phenyl group, a naphthyl group, a phenoxy group, or a naphthyloxy group binds to the above aryl groups.

Examples of the aryloxy group which may have a substituent include an aryloxy group having 6 to 10 carbon atoms such as phenoxy or naphthyloxy; and an aryloxy group in which a substituent such as a fluorine atom, a hydroxyl group, a cyano group, an amino group, a methoxy group, an ethoxy group, an isopropyloxy group, a phenyl group, a naphthyl group, a phenoxy group, or a naphthyloxy group binds to the above aryloxy groups.

Examples of the acyl group which may have a substituent include an acyl group having 2 to 20 carbon atoms such as acetyl, propionyl, butyryl, isobutyryl, benzoyl, 1-naphthoyl, or 2-naphthoyl; and an acyl group in which a substituent such as a fluorine atom, a hydroxyl group, a cyano group, an amino group, a methoxy group, an ethoxy group, an isopropyloxy group, a phenyl group, a naphthyl group, a phenoxy group, or a naphthyloxy group binds to the above acyl groups.

Y¹ and Y² each independently represents a single bond, a carbonyl group, a sulfonyl group, an isopropylidene group, a hexafluoroisopropylidene group, or a fuorene-9,9-diyl group. Z¹ and Z₂ each independently represents an oxygen atom or a sulfur atom.

The polystyrene-equivalent weight average molecular weight of the structural unit represented by the formula (5) is, for example, in a range of from 1,000 to 2,000,000, and preferably is, for example, in a range of from 1,000 to 500,000. When the polymer of the present invention is used as a polymer electrolyte for a solid polymer fuel cell, the polystyrene-equivalent weight average molecular weight preferably is, for example, in a range of from 2,000 to 2,000,000, and more preferably is, for example, in a range of from 2,000 to 1,000,000, and even more preferably is, for example, in a range of from 3,000 to 800,000.

Specific examples of the structural unit represented by the formula (5) include structural units represented by the following the formulae (5a) to (5z). In the following formulae, n has the same definition as those described above. Specifically, examples of n include an integer in a range of, for example, from 2 to 200, and preferably in a range of, for example, from 5 to 200. The polystyrene-equivalent weight average molecular weight of the structural unit represented by the formula (5) is, for example, 1,000 or more, preferably is, for example, 2,000 or more, and more preferably is, for example, 3,000 or more.

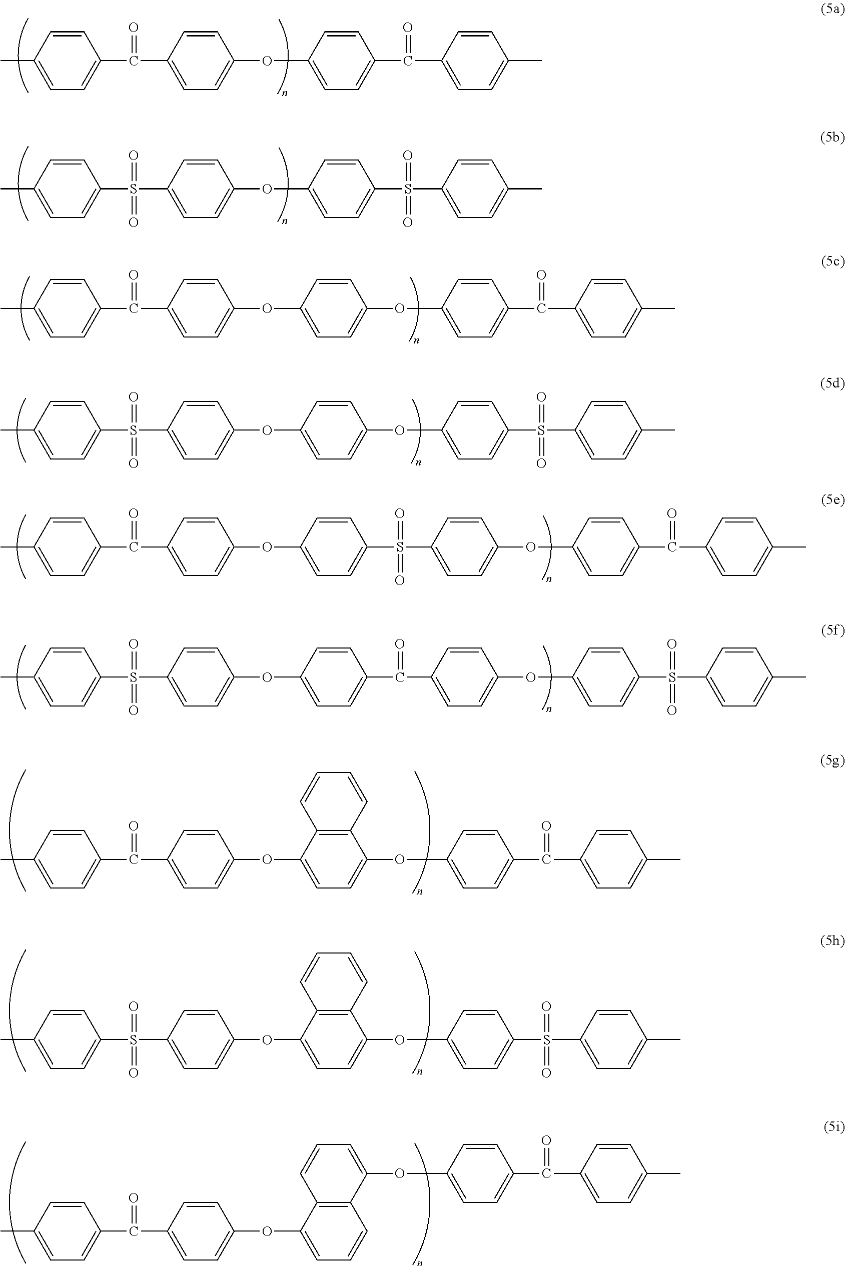

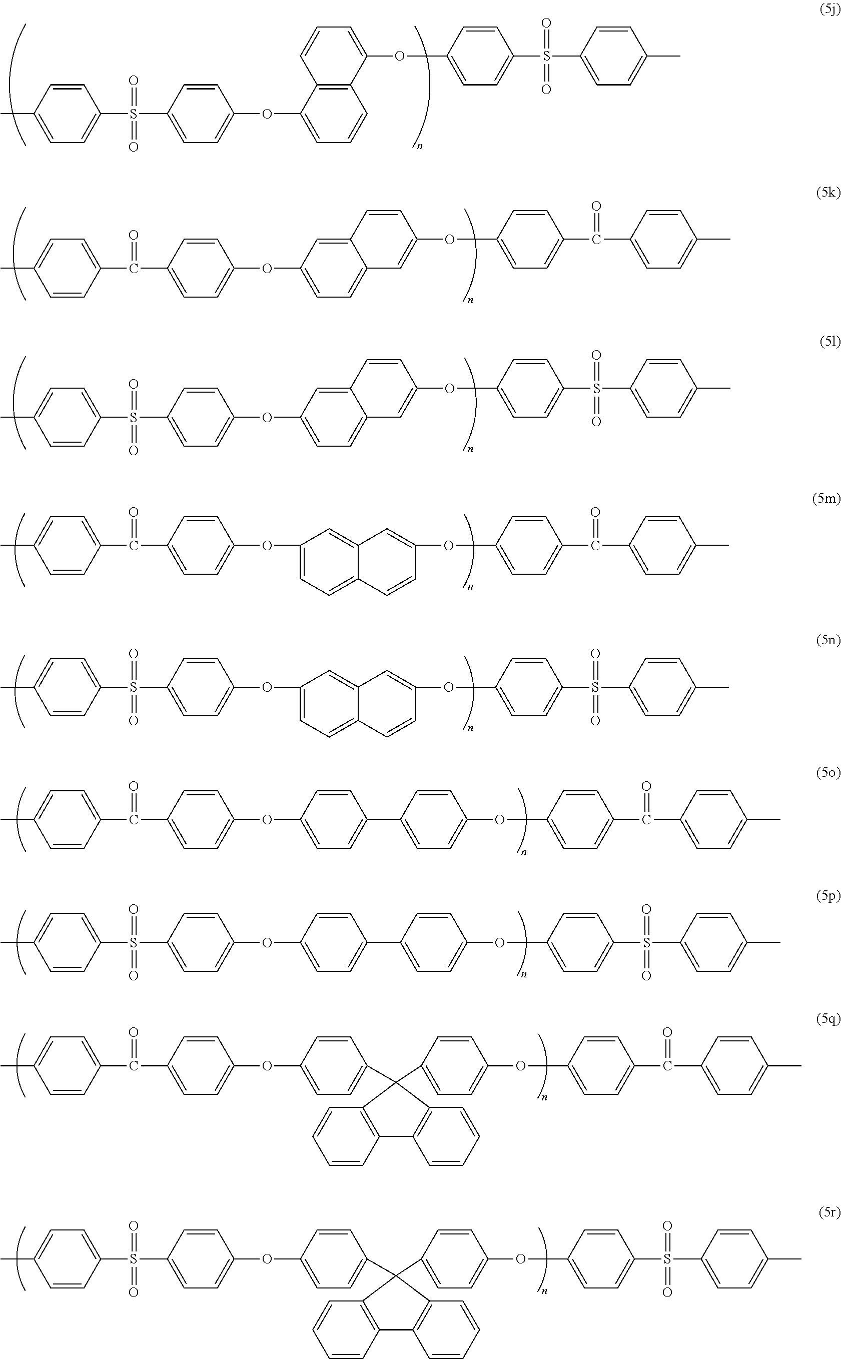

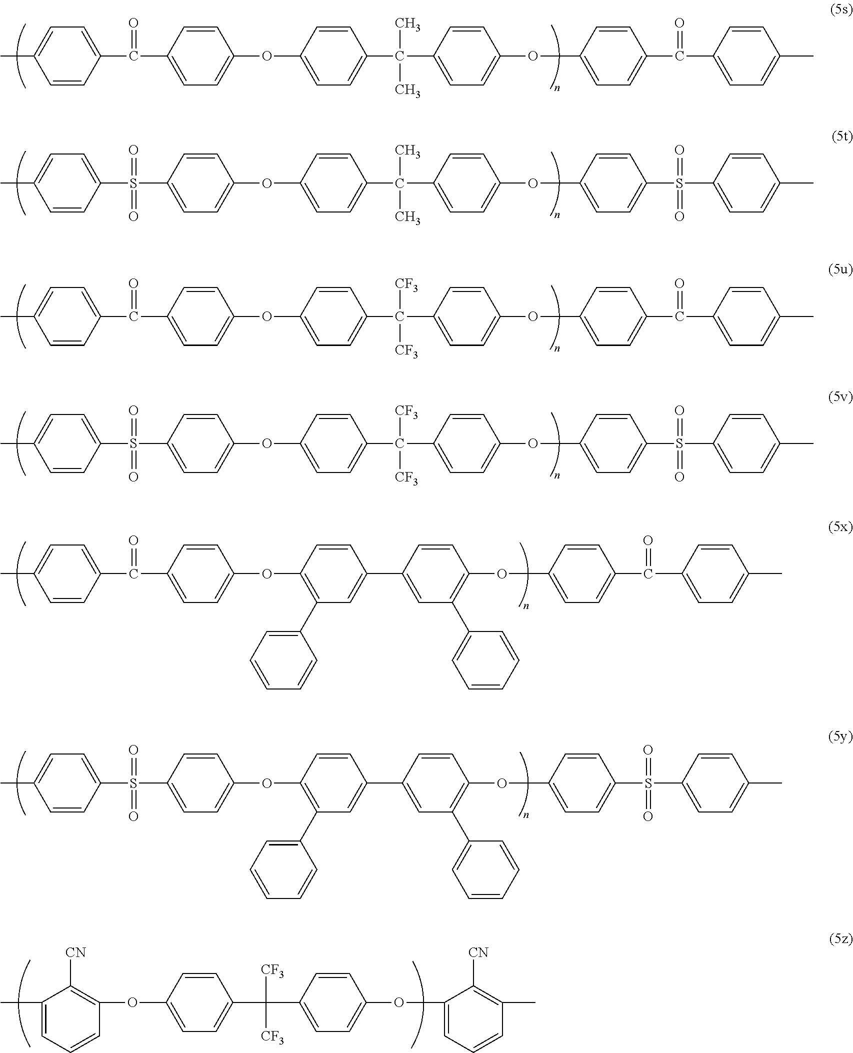

Examples of the macromolecule having the structural unit represented by the formula (5) include a macromolecule (hereinafter, abbreviated to a macromolecule (6)) which has groups at both ends of the structural unit represented by the formula (5) those are able to form a bond by reacting with X¹ of the biphenyltetrasulfonic acid compound represented by the formula (1), and which has elimination groups such as halogen atoms at both ends of the macromolecule. Examples of the method for producing the macromolecule (6) include methods disclosed in JP-2003-113136-A and JP-2007-138065-A.

The preferable polystyrene-equivalent weight average molecular weight of the macromolecule (6) is, for example, 1,000 or more, and preferably is, for example 2,000 or more, and more preferably is, for example, 3,000 or more.

As the macromolecule (6), commercially available ones may be used, and examples of the commercially available macromolecule (6) include SUMIKAEXCEL (manufactured by Sumitomo Chemical Company, registered trademark) PES 3600P, 4100P, 4800P, and 5200P.

Examples of the method of polymerizing the compound (Y) and/or the macromolecule (6) with the biphenyltetrasulfonic acid compound represented by the formula (1) include a method of polymerizing a composition containing the compound (Y) and/or the macromolecule (6) and the biphenyltetrasulfonic acid compound represented by the formula (1) in the presence of a nickel compound, and a method of polymerizing the biphenyltetrasulfonic acid compound represented by the formula (1) in the presence of a nickel compound and then further polymerizing after adding the compound (Y) and/or the macromolecule (6) thereto.

Examples of the nickel compound used in the above method include a zerovalent nickel compound such as nickel (0) bis(cyclooctadiene), nickel (0) (ethylene)bis(triphenylphosphine), or nickel (0) tetrakis(triphenylphosphine); and a divalent nickel compound such as nickel halide (for example, nickel fluoride, nickel chloride, nickel bromide, or nickel iodide), a nickel carboxylic acid salt (for example, nickel formate or nickel acetate), nickel sulfate, nickel carbonate, nickel nitrate, nickel acetylacetonate, or (dimethoxyethane) nickel chloride. Among these, nickel (0) bis(cyclooctadiene) and nickel halide are preferable.

The amount of the nickel compound used is, for example, in a range of from 0.01 mol to 5 mol, based on the total molar amount of the biphenyltetrasulfonic acid compound represented by the formula (1), the compound (Y), and the macromolecule (6).

When the polymerization is performed using the divalent nickel compound as a catalyst, it is preferable to perform the polymerization in the presence of a nitrogen-containing bidentate ligand. Examples of the nitrogen-containing bidentate ligand include 2,2′-bipyridine, 1,10-phenanthroline, methylenebisoxazoline and N,N,N′,N′-tetramethylethylenediamine, and among these, 2,2′-bipyridine is preferable. When the nitrogen-containing bidentate ligand is used, the amount thereof used is in a range of, for example, from 0.2 mol to 2 mol, and preferably is in a range of, for example, from 1 mol to 1.5 mol, based on 1 mol of the nickel compound.

When the polymerization is performed using the divalent nickel compound as a catalyst, it is preferable to use zinc concurrently, and generally, powdered zinc is used. When zinc is used, the amount thereof used is, for example, in a range of from 0.5 times to 1.5 times the total molar amount of the biphenyltetrasulfonic acid compound represented by the formula (1), the compound (Y), and the macromolecule (6).

It is preferable to perform the polymerization reaction in the presence of a solvent. As the solvent, a solvent that can dissolve the biphenyltetrasulfonic acid compound represented by the formula (1), the compound (Y), and the macromolecule (6) as well as the obtaining polymer may be used. Specific examples of the solvent include an aromatic hydrocarbon solvent such as toluene or xylene; an ether solvent such as tetrahydrofuran or 1,4-dioxane; an aprotonic polar solvent such as dimethyl sulfoxide, N-methyl-2-pyrrolidone, N,N-dimethylformamide, N,N-dimethylacetamide, or hexamethylphosphoric triamide; and a halogenated hydrocarbon solvent such as dichloromethane or dichloroethane. These solvents may be used alone or as a mixture of two or more kinds thereof. Among these, an ether solvent and an aprotonic polar solvent are preferable, and tetrahydrofuran, dimethyl sulfoxide, N-methyl-2-pyrrolidone, and N,N-dimethylacetamide are more preferable.

The amount of the solvent used is generally 1 time to 200 times by weight, and preferably 5 times to 100 times by weight of the total weight of the biphenyltetrasulfonic acid compound represented by the formula (1), the compound (Y), and the macromolecule (6). If the amount is 1 time by weight or more, a polymer of a large molecular weight tends to be easily obtained, and if the amount is 200 times by weight or less, operability during the polymerization and operability such as taking out the polymer after the completion of polymerization reaction tend to be excellent.

It is preferable to perform the polymerization reaction in an atmosphere of inert gas such as nitrogen gas.

The reaction temperature of the polymerization reaction is, for example, in a range of from 0° C. to 250° C., and preferably is in a range of from 30° C. to 100° C. The polymerization time is, for example, in a range of from 0.5 hours to 48 hours.

After the completion of the polymerization reaction, a solvent that poorly dissolves the generated polymer is mixed with the reaction mixture so as to precipitate the polymer, and the precipitated polymer is separated from the reaction mixture through filtering, whereby the polymer of the present invention can be taken out.

A solvent that does not dissolve or poorly dissolves the generated polymer may be mixed with the reaction mixture, and then an acid may be added thereto so as to separate the precipitated polymer from the reaction mixture through filtering.

Examples of the solvent that does not dissolve or poorly dissolves the generated polymer include water, methanol, ethanol, and acetonitrile. Among these, water and methanol are preferable.

Examples of the acid include hydrochloric acid and sulfuric acid. The amount of the acid used may be an amount sufficient for acidifying the reaction mixture.

Examples of a preferable polymer include a polymer comprising a structural unit originating from the biphenyltetrasulfonic acid compound represented by the formula (1).

Examples of the method of polymerizing the biphenyltetrasulfonic acid compound represented by the formula (1) include a method of polymerizing a composition containing the biphenyltetrasulfonic acid compound represented by the formula (1) in the presence of a nickel compound.

Examples of the nickel compound include a zerovalent nickel compound such as nickel (0) bis(cyclooctadiene), nickel (0) (ethylene)bis(triphenylphosphine), or nickel (0) tetrakis(triphenylphosphine); and a divalent nickel compound such as nickel halide (for example, nickel fluoride, nickel chloride, nickel bromide, or nickel iodide), a nickel carboxylic acid salt (for example, nickel formate or nickel acetate), nickel sulfate, nickel carbonate, nickel nitrate, nickel acetylacetonate, or (dimethoxyethane) nickel chloride. Among these, nickel (0) bis(cyclooctadiene) and nickel halide are preferable.

The amount of the nickel compound used is, for example, in a range of from 0.01 mol to 5 mol, based on 1 mol of the biphenyltetrasulfonic acid compound represented by the formula (1).

When the polymerization is performed using the divalent nickel compound as a catalyst, it is preferable to perform the polymerization in the presence of a nitrogen-containing bidentate ligand. Examples of the nitrogen-containing bidentate ligand include 2,2′-bipyridine, 1,10-phenanthroline, methylenebisoxazoline and N,N,N′,N′-tetramethylethylenediamine, and among these, 2,2′-bipyridine is preferable. When the nitrogen-containing bidentate ligand is used, the amount thereof used is in a range of, for example, from 0.2 mol to 2 mol, and preferably is in a range of, for example, from 1 mol to 1.5 mol, based on 1 mol of the nickel compound.

When the polymerization is performed using the divalent nickel compound as a catalyst, it is preferable to use zinc concurrently, and generally, powdered zinc is used. When zinc is used, the amount thereof used is, for example, in a range of from 0.5 mol to 1.5 mol, based on 1 mol of the biphenyltetrasulfonic acid compound represented by the formula (1).

It is preferable to perform the polymerization reaction in the presence of a solvent. As the solvent, a solvent that can dissolve the biphenyltetrasulfonic acid compound represented by the formula (1) and the obtaining polymer may be used. Specific examples of the solvent include an aromatic hydrocarbon solvent such as toluene or xylene; an ether solvent such as tetrahydrofuran or 1,4-dioxane; an aprotonic polar solvent such as dimethyl sulfoxide, N-methyl-2-pyrrolidone, N,N-dimethylformamide, N,N-dimethylacetamide, or hexamethylphosphoric triamide; and a halogenated hydrocarbon solvent such as dichloromethane or dichloroethane. These solvents may be used alone or as a mixture of two or more kinds thereof. Among these, an ether solvent and an aprotonic polar solvent are preferable, and tetrahydrofuran, dimethyl sulfoxide, N-methyl-2-pyrrolidone, and N,N-dimethylacetamide are more preferable.

The amount of the solvent used is generally 1 time to 200 times by weight, and preferably 5 times to 100 times by weight of the amount of the biphenyltetrasulfonic acid compound represented by the formula (1) used. If the amount is 1 time by weight or more, a polymer of a large molecular weight tends to be easily obtained, and if the amount is 200 times by weight or less, operability during the polymerization and operability such as taking out the polymer after the completion of polymerization reaction tend to be excellent.

It is preferable to perform the polymerization reaction in an atmosphere of inert gas such as nitrogen gas.

The reaction temperature of the polymerization reaction is, for example, in a range of from 0° C. to 250° C., and preferably is in a range of from 30° C. to 100° C. The polymerization time is, for example, in a range of from 0.5 hours to 48 hours.

After the completion of the polymerization reaction, a solvent that poorly dissolves the generated polymer is mixed with the reaction mixture so as to precipitate the polymer, and the precipitated polymer is separated from the reaction mixture through filtering, whereby the polymer of the present invention can be taken out.

A solvent that does not dissolve or poorly dissolves the generated polymer may be mixed with the reaction mixture, and then an acid may be added thereto so as to separate the precipitated polymer from the reaction mixture through filtering.

Examples of the solvent that does not dissolve or poorly dissolves the generated polymer include water, methanol, ethanol, and acetonitrile. Among these, water and methanol are preferable.

Examples of the acid include hydrochloric acid and sulfuric acid. The amount of the acid used may be an amount sufficient for acidifying the reaction mixture.

When the structural unit, which originates from the biphenyltetrasulfonic acid compound represented by the formula (1), of the obtained polymer contains R¹O—, and R¹ is a hydrocarbon group, it is necessary to make R¹ into a hydrogen atom or a cation by performing a deprotection reaction. The deprotection reaction is performed based on, for example, a method disclosed in JP-2007-270118-A.

The value of an ion exchange capacity (measured by a titration method) of the polymer obtained in this manner is in a range of, for example, from 0.5 meq/g to 8.0 meq/g, and preferably is in a range of, for example, from 0.5 meq/g to 6.5 meq/g.

The molecular weight and structure of the obtained polymer can be analyzed by general analysis means such as gel permeation chromatography or NMR.

All of the polymers obtained in this manner can be suitably used as a member for fuel cells. The polymer of the present invention is used preferably as a polymer electrolyte of an electrochemical device such as a fuel cell, and particularly preferably as a polymer electrolyte membrane. That is, the polymer electrolyte of the present invention contains the polymer of the present invention, and the polymer electrolyte membrane of the present invention contains the polymer electrolyte of the present invention. Hereinafter, a description will be made focusing mainly on a case of this polymer electrolyte membrane.

In this case, the polymer electrolyte of the present invention is formed into a membrane. There is no particular limitation on this method (membrane-forming method), but it is preferable to form a membrane by using a method of forming a membrane in a solution state (a solution casting method). The solution casting method is a method which has been widely used in the field of the related art to produce a polymer electrolyte membrane, and is useful industrially in particular.

Specifically, the polymer electrolyte of the present invention is dissolved in an appropriate solvent to prepare a polymer electrolyte solution, and this polymer electrolyte solution is cast onto a support substrate, followed by removal of the solvent, thereby forming a membrane. Examples of the support substrate include a glass plate and plastic films such as polyethylene (PE), polypropylene (PP), polyethylene terephthalate (PET), polyethylene naphthalate (PEN), and polyimide (PI).

The solvent (cast solvent) used in the solution casting method is not particularly limited as far as the solvent can sufficiently dissolve the polymer electrolyte of the present invention and is removable after the membrane is formed by the casting solution method. As the solvent, an aprotonic polar solvent such as N-methyl-2-pyrrolidone (NMP), N,N-dimethylacetamide (DMAc), N-dimethylformamide (DMF), 1,3-dimethyl-2-imidazolidinone (DMI), or dimethyl sulfoxide (DMSO); a chlorine-based solvent such as dichloromethane, chloroform, 1,2-dichloroethane, chlorobenzene, or dichlorobenzene; alcohols such as methanol, ethanol, and propanol; and alkylene glycol monoalkyl ethers such as ethylene glycol monomethyl ether, ethylene glycol monoethyl ether, propylene glycol monomethyl ether, or propylene glycol monoethyl ether can be suitably used. These solvents may be used alone or optionally as a mixture of two or more kinds of solvents. Among these, NMP, DMAc, DMF, DMI, and DMSO are preferable since the polymer electrolyte of the present invention exhibit high solubility in these solvents and a polymer electrolyte membrane with high water resistance is obtained if these solvents are used.

The polymer electrolyte membrane obtained in this manner is excellent in a water vapor permeability. That is, in this polymer electrolyte membrane, a parameter value defined by [(water vapor permeability coefficient)/(weight fraction of structural unit having sulfonic acid group relative to the polymer)] is larger than that of a conventional hydrocarbon-based polymer electrolyte. The value of the weight fraction of a structural unit having a sulfonic acid group relative to the polymer constituting the polymer electrolyte membrane is in a range of, for example, from 0.05 to 0.85, preferably is in a range of, for example, from 0.10 to 0.80, and even more preferably is in a range of, for example, from 0.15 to 0.75. If the weight fraction of the structural unit having a sulfonic acid group relative to the polymer constituting the polymer electrolyte membrane is 0.05 or more, a power generation performance tends to be improved, and if it is 0.90 or less, water resistance tends to be improved. The water vapor permeability coefficient of the polymer electrolyte membrane is, for example, 3.0×10⁻¹⁰ mol/sec/cm or more, preferably is, for example, 4.0×10⁻¹⁰ mol/sec/cm or more, and more preferably is, for example, 5.0×10⁻¹⁰ mol/sec/cm or more. If the water vapor permeability coefficient of the polymer electrolyte membrane is 3.0×10⁻¹⁰ mol/sec/cm or more, the power generation performance tends to be improved. In addition, a value that is obtained by dividing the water vapor permeability coefficient of the polymer electrolyte membrane by the weight fraction of the structural unit having a sulfonic acid group relative to the polymer constituting the polymer electrolyte membrane is, for example, 2.0×10⁻⁹ mol/sec/cm or more, preferably is, for example, 2.2×10⁻⁹ mol/sec/cm or more, and more preferably is, for example, 2.4×10⁻⁹ mol/sec/cm or more.

In producing the polymer constituting the polymer electrolyte membrane, by controlling the ratio between the biphenyltetrasulfonic acid compound represented by the formula (1) and the compound (Y) and/or the macromolecule (6) incorporated, a polymer electrolyte membrane having a desired water vapor permeability coefficient can be obtained.

Although the thickness of the polymer electrolyte membrane obtained in this manner is not particularly limited, the thickness is preferably in a range of from 5 μm to 300 μm which is practical as the thickness of the polymer electrolyte membrane (thin membrane) for a fuel cell. A membrane having a thickness of 5 μm or more is excellent in practical strength, and in a membrane having a thickness of 300 μm or less, membrane resistance itself tends to be decreased. The membrane thickness can be controlled by the concentration of the solution described above and the thickness of the solution applied to the membrane on the support substrate.

In addition, for the purpose of improving various properties of the membrane, additives such as a plasticizer, a stabilizer, and a release agent that are used for general macromolecules may be added to the polymer of the present invention to prepare the polymer electrolyte. Moreover, it is also possible to prepare the polymer electrolyte by making a composite alloy of the copolymer of the present invention and another polymer, through a method of casting together by mixing these components in the same solution. In this way, when the polymer of the present invention, additives and/or another polymer are combined together to prepare the polymer electrolyte, the type and used amount of the additives and/or another polymer are determined so that desired properties may be obtained when the polymer electrolyte is applied to a member for a fuel cell.

It is also known that for the use of the polymer electrolyte for a fuel cell, in order to effectively use water generated in the fuel cell, inorganic or organic fine particles are added as a water retention agent. These any well-known method can be used as far as the method does not impede the object of the present invention. In addition, for the purpose of, for example, improving mechanical strength of the polymer electrolyte membrane obtained in this manner, a treatment such as electron beam irradiation, radiation, or the like may be carried out.

For the purpose of further improving the strength, flexibility, and durability of the polymer electrolyte membrane containing the polymer electrolyte of the present invention, it is effective to form a polymer electrolyte composite membrane having the polymer electrolyte of the present invention and a porous substrate. The polymer electrolyte composite membrane (hereinafter, also referred to as a “composite membrane”) can be formed by impregnating the porous substrate with the polymer electrolyte of the present invention so as to make a composite membrane. As the method of forming a composite membrane, known methods can be used.

The porous substrate is not particularly limited as far as the substrate is suitable for the usage purpose described above, and examples thereof include a porous membrane, woven fabric, and nonwoven fabric. The porous substrate can be used regardless of the shape or material thereof as far as the substrate is suitable for the usage purpose described above. As the material of the porous substrate, an aliphatic macromolecule and an aromatic macromolecule are preferable, from the viewpoint of heat resistance and in consideration of an effect of reinforcing physical strength.

When the composite membrane containing the polymer electrolyte of the present invention is used as a polymer electrolyte membrane, the membrane thickness of the porous substrate is preferably 1 μm to 100 μm, more preferably 3 μm to 30 μm, and particularly preferably 5 μm to 20 μm. The pore size of the porous substrate is preferably 0.01 μm to 100 μm, and more preferably 0.02 μm to 10 μm. The porosity of the porous substrate is preferably 20% to 98%, and more preferably 40% to 95%.

If the membrane thickness of the porous substrate is 1 μm or more, the strength-reinforcing effect obtained by making a composite membrane and the reinforcing effect that imparts flexibility or durability become superior, and gas leakage (cross leakage) does not easily occur. In addition, if the membrane thickness is 100 μm or less, electric resistance is further reduced, and the obtained composite membrane becomes more excellent as a polymer electrolyte membrane for a fuel cell. If the pore size is 0.01 μm or more, the polymer of the present invention is more easily filled in the pore, and if the pore size is 100 μm or less, the reinforcing effect is further enhanced. If the porosity is 20% or more, resistance of the polymer electrolyte membrane is further reduced, and if the porosity is 98% or less, the strength of the porous substrate itself is further increased, whereby the reinforcing effect is further improved.

It is also possible to form a proton conductive membrane by laminating the polymer electrolyte composite membrane of the present invention with the polymer electrolyte membrane of the present invention.

Next, the fuel cell of the present invention will be described.

A membrane electrode assembly (hereinafter sometimes referred to as “MEA”) of the present invention that serves as a basic unit of a fuel cell contains at least one kind selected from the group consisting of the polymer electrolyte membrane of the present invention, the polymer electrolyte composite membrane of the present invention, and a catalyst composition comprising the polymer electrolyte of the present invention and a catalyst component. The membrane-electron assembly can be produced using at least one kind of this material.

The catalyst component is not particularly limited as far as the component is a substance that can activate an oxidation-reduction reaction with hydrogen or oxygen. Although known substances can be used as the catalyst component, it is preferable use fine particles of platinum or a platinum-based alloy as the catalyst component. In some cases, the fine particles of platinum or a platinum-based alloy are used by being supported frequently on particle-like or fiber-like carbon such as activated carbon or graphite.

The platinum or platinum-based alloy supported on carbon (carbon-supported catalyst) is mixed with a solution of the polymer electrolyte of the present invention and/or an alcohol solution of a perfluoroalkylsulfonic acid resin as a polymer electrolyte and made into a paste so as to obtain a catalyst composition, and the composition is applied to a gas diffusion layer and/or a polymer electrolyte membrane and/or a polymer electrolyte composite membrane, followed by drying, whereby a catalyst layer is obtained. As a specific method thereof, for example, a well-known method such as a method disclosed in J. Electrochem. Soc.: Electrochemical Science and Technology, 1988, 135(9), 2209 can be used. In this manner, by forming a catalyst layer on both surfaces of the polymer electrolyte membrane, the MEA of the present invention is obtained. In producing the MEA, when the catalyst layer is formed on the substrate as the gas diffusion layer, the MEA is obtained in the form of an assembly of membrane-electrode-gas diffusion layer that includes both the gas diffusion layer and the catalyst layer on both surfaces of the polymer electrolyte membrane. In addition, when the catalyst composition made into a paste is applied to the polymer electrolyte membrane and dried to form a catalyst layer on the polymer electrolyte membrane, a gas diffusion layer is further formed on the obtained catalyst layer, whereby an assembly of membrane-electrode-gas diffusion layer is obtained.

Although well-known materials can be used for the gas diffusion layer, in order to efficiently transport raw material gas to a catalyst, porous woven carbon fabric, nonwoven carbon fabric, or carbon paper is preferable.

The polymer electrolyte fuel cell including the MEA of the present invention produced in this manner is usable not only in the form of using hydrogen gas or modified hydrogen gas as fuel, but also in various forms of using methanol.

### EXAMPLES

Hereinafter, the present invention will be described in more detail based on examples.

The polymer described in Example 4 was analyzed (the analysis conditions were as follows) by gel permeation chromatography (hereinafter, abbreviated to GPC), and from the analysis results, the polystyrene-equivalent weight average molecular weight (Mw) and the polystyrene-equivalent number average molecular weight (Mn) were calculated.

<Analysis Conditions 1>

GPC measurement instrument: CTO-10A (manufactured by Shimadzu Corporation.)

Column: TSK-GEL GMHHR-M (manufactured by TOSOH CORPORATION)

Column temperature: 40° C.

Mobile phase: lithium bromide-containing N,N-dimethylacetamide (lithium bromide concentration: 10 mmol/dm³)

Flow rate: 0.5 mL/min

Detection wavelength: 300 nm

The polymers described in Examples 5 to 8 were analyzed by GPC (the analysis conditions were as follows), and from the analysis results, the polystyrene-equivalent Mw and Mn were calculated.

<Analysis Conditions 2>

GPC measurement instrument: Prominence GPC system (manufactured by Shimadzu Corporation.)

Column: TSKgel GMH_{HR}-M (manufactured by TOSOH CORPORATION)

Column temperature: 40° C.

Mobile phase: lithium bromide-containing DMF (lithium bromide concentration: 10 mmol/dm³)

Flow rate: 0.5 mL/min

Detection: differential refractive index

Ion exchange capacity (IEC) measurement:

The polymer (a polymer electrolyte) to be provided to the measurement was formed into a membrane by the solution casting method to obtain a polymer electrolyte membrane, and the obtained polymer electrolyte membrane was cut so as to yield an appropriate weight. The dry weight of the cut polymer electrolyte membrane was measured using a halogen moisture meter set at a heating temperature of 105° C. Thereafter, the polymer electrolyte membrane dried in this manner was dipped in 5 mL of a 0.1 mol/L aqueous sodium hydroxide solution, 50 ml of ion exchange water was then further added thereto, and the resultant was left for 2 hours. Subsequently, titration was performed by slowly adding 0.1 mol/L hydrochloric acid to the solution in which the polymer electrolyte membrane was dipped, and a point of neutralization was determined. Next, from the dry weight of the cut polymer electrolyte membrane and the amount of the hydrochloric acid required for neutralization, the ion exchange capacity (unit: meq/g) of the polymer electrolyte was calculated.

Water vapor permeability measurement:

At both sides of the polymer electrolyte membrane, a separator (gas flowing area of 1.3 cm²) made of carbon for a fuel cell, in which a groove as a passage for gas had been cut, was disposed. Furthermore, a current collector and an endplate were arranged in order in the outside of the separator, and the resultant was tightened up by a bolt, thereby assembling a cell for water vapor permeability measurement. Between the polymer electrolyte membrane and the separator made of carbon, a silicon gasket having 1.3 cm² of an opening portion with the same shape as that of the gas flowing portion of the separator was disposed.

The cell temperature was set to 85° C., hydrogen gas with a relative humidity of 20% was allowed to flow through one side of the cell at a flow rate of 1000 mL/min, and air with a relative humidity of about 0% was allowed to flow through the other side of the cell at a flow rate of 200 mL/min. The backpressure of the both sides was set to 0.04 MPaG. A dew-point meter was disposed at the air outlet side of the cell to measure a dew point of the gas at the outlet, whereby the amount of moisture contained in the air at the outlet was measured and a water vapor permeability coefficient [mol/sec/cm] was calculated.

### Example 1

### Synthesis of disodium 1-bromo-4-chloro-2,6-benzenedisulfonate

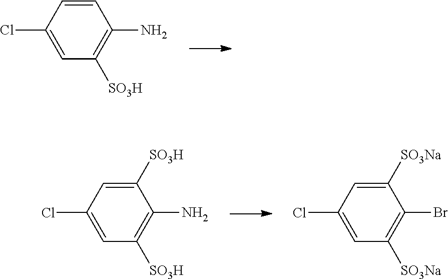

Commercially available 2-amino-5-chlorobenzenesulfonic acid (53.0 g) was slowly added to 265.0 g of 30% fuming sulfuric acid, at 25° C., and the temperature of the obtained mixture was raised up to 120° C. and kept at this temperature for 2 hours. The reaction mixture was poured into 265.0 g of cold water, 74.0 g of a 36% aqueous sodium nitrite solution was slowly added dropwise thereto at 10° C., and the obtained mixture was kept at this temperature for 1 hour. The obtained mixture was called a “diazo mass 1”. Meanwhile, 74.0 g of monovalent copper bromide was dissolved in 369.9 g of 48% hydrobromic acid, and the temperature was raised up to 35° C. The entire “diazo mass 1” was added dropwise to the obtained mixture, over 30 minutes, and the obtained mixture was kept at this temperature for 1 hour. The reaction mixture was cooled to −10° C., followed by filtering, and the obtained solid and 976.8 g of water were mixed. Thereafter, 10.7 g of a 50% aqueous sodium hydroxide solution was added thereto, and the precipitated solid was filtered. The filtrate was adjusted to pH 6 by using concentrated hydrochloric acid, followed by concentration and drying, thereby obtaining 72.5 g (yield 71.8%) white solid disodium 1-bromo-4-chloro-2,6-benzenedisulfonate (which is referred to as a “product 1”).

¹H-NMR (heavy water, δ (ppm)): 8.11 (s, 2H)

### Example 2

### Synthesis of tetrasodium 4,4′-dichloro-2,2′,6,6′-biphenyltetrasulfonate

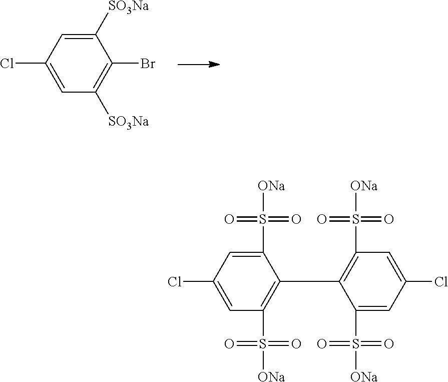

N,N-dimethylformamide (579.6 g) was added to 72.5 g of the product 1 (disodium 1-bromo-4-chloro-2,6-benzenedisulfonate) synthesized in Example 1, followed by heating at 100° C. to dissolve the product 1. Thereafter, the resultant was subjected to vacuum concentration, thereby distilling away 395.5 g of N,N-dimethylformamide. The value of moisture contained in the concentrated mass was 276 ppm. After the concentrated mass was cooled to 25° C., 23.4 g of copper powder, 17.4 g of monovalent copper iodide, and 101.7 g of N,N-dimethylformamide were added to the concentrated mass, and the temperature of the obtained mixture was raised up to 150° C. and kept at this temperature for 2 hours. The reaction mixture was poured into 1156.3 g of water, followed by filtration of insoluble matter, and the filtrate was concentrated and dried. The concentrated substance was dissolved in 193.2 g of water, 391.4 g of 2-propanol was slowly added thereto, and the precipitated solid was filtered and dried, thereby obtaining 44.0 g (yield 76.1%) of white solid tetrasodium 4,4′-dichloro-2,2′,6,6′-biphenyltetrasulfonate.

¹H-NMR (heavy DMSO, δ (ppm)): 7.23 (s, 2H)

Mass spectrum (ESI, m/z): 541 (M⁻¹)

Element analysis: Na (15.1%)

### Example 3

### Synthesis of tris(2,2-dimethyl-1-propyl) sodium 4,4′-dichloro-2,2′,6,6′-biphenyltetrasulfonate

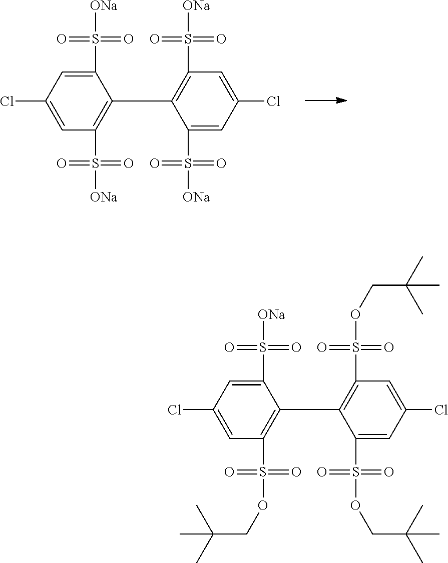

Chloroform (300.0 g), 3.5 g of N,N-dimethylformamide and 33.9 g of thionyl chloride were added to 15.0 g of tetrasodium 4,4′-dichloro-2,2′,6,6′-biphenyltetrasulfonate synthesized in Example 2, and the temperature of the obtained mixture was raised up to 55° C. and kept at this temperature for 1 hour, followed by concentration and drying of the reaction mixture. The obtained concentrated residue was called a “concentrate 1”. Meanwhile, a 1.65 M hexane solution of n-butyllithium (115.2 mL, 190 mmol) was added dropwise to a solution including 20.9 g of 2,2-dimethyl-1-propanol and 146.6 g of anhydrous tetrahydrofuran, at 25° C., and the resultant was kept at this temperature for 30 minutes. The “concentrate 1” was incorporated to this solution, and the resultant was kept at 25° C. for 14 hours. The reaction mixture was poured into a solution including 276.5 g of toluene and 276.5 g of water, and the water layer was removed. The organic layer was washed with 237.8 g of a 5% aqueous sodium carbonate solution, followed by drying over sodium sulfate, and the resultant was concentrated and dried. The concentrated residue was purified with silica gel chromatography (mobile phase: ethyl acetate), and the obtained eluate was washed with 276.5 g of 5% aqueous sodium carbonate solution. The resultant was dried over sodium sulfate, and concentrated and dried. The concentrate was washed with a mixed solvent including 21.0 g of toluene and 156.0 g of hexane, and the solid obtained after filtration was dried, thereby obtaining 7.0 g (yield 38.0%) of white solid tris(2,2-dimethyl-1-propyl) sodium 4,4′-dichloro-2,2′,6,6′-biphenyltetrasulfonate.

¹H-NMR (heavy chloroform, δ (ppm)): 0.97 (s, 27H), 3.83-4.04 (c, 6H), 7.82 (d, 1H), 8.00 (s, 2H), 8.36 (s, 1H)

Mass spectrum (ESI, m/z): 752 (M⁻¹)

Element analysis: C (43.5%), H (5.3%), S (15.8%), Cl (8.7%), Na (2.9%)

### Example 4

### Polymer Synthesis

The temperature of a solution which contained 0.75 g (0.97 mmol) of tris(2,2-dimethyl-1-propyl) sodium 4,4′-dichloro-2,2′,6,6′-biphenyltetrasulfonate obtained in Example 3, 0.77 g of SUMIKAEXCEL (manufactured by Sumitomo Chemical Company, registered trademark) PES 3600P (Mn=2.7×10⁴, Mw=4.4×10⁴) having a structure represented by the following formula:

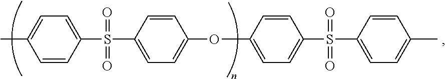

0.755 g of 2,2′-bipyridine, and 11.3 g of dimethyl sulfoxide was raised up to 70° C., and 1.33 g of nickel (0) bis(cyclooctadiene) was added thereto, followed by stirring for 4 hours. The obtained reaction mixture was poured into 74.3 g of a 25% aqueous nitric acid solution, the precipitate was filtered, and a cake obtained by the filtration was washed three times with water. Anhydrous lithium bromide (1.34 g) and 22.8 g of N-methyl-2-pyrrolidone were added to the washed cake, and the obtained mixture was stirred at 120° C. for 4 hours.

The obtained mixture was poured into 150.0 g of 19% hydrochloric acid, and crystals were precipitated, followed by filtration. The obtained cake was washed with water and dried, thereby obtaining 0.98 g of a polymer having a structural unit originating from 4,4′-dichloro-2,2′,6,6′-biphenyltetrasulfonic acid. Mw of the obtained polymer was 7.0×10⁴, Mn thereof was 2.5×10⁴, and the ion exchange capacity thereof was 1.92 meq/g.

### Example 5

### Polymer Synthesis

The temperature of a solution which contained 0.56 g (0.72 mmol) of tris(2,2-dimethyl-1-propyl) sodium 4,4′-dichloro-2,2′,6,6′-biphenyltetrasulfonate obtained in Example 3, 0.53 g (2.11 mmol) of 2,5-dichlorobenzophenone, 2.33 g of 2,2′-bipyridine, and 32 g of DMSO was raised up to 60° C., and 3.90 g of nickel (0) bis(cyclooctadiene) was added thereto, followed by stirring for 5 hours. The obtained reaction mixture was poured into 150 g of a 25% aqueous nitric acid solution, followed by filtration of the precipitate, and a cake obtained by the filtration was washed three times with water. Anhydrous lithium bromide (0.75 g) and 9 g of N-methyl-2-pyrrolidone were added to the washed cake, and the obtained mixture was stirred at 120° C. for 24 hours.

The obtained mixture was poured into 100 g of 19% hydrochloric acid, and crystals were precipitated, followed by filtration. The obtained cake was washed with water and dried, thereby obtaining 0.41 g of a polymer having a structural unit originating from 4,4′-dichloro-2,2′,6,6′-biphenyltetrasulfonic acid shown below. Mw of the obtained polymer was 6.3×10⁴, and Mn thereof was 2.6×10⁴. The obtained polymer was water-insoluble.

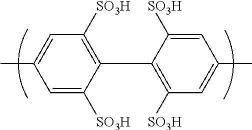

### Example 6

### Polymer Synthesis

The temperature of a solution which contained 1.05 g (1.35 mmol) of tris(2,2-dimethyl-1-propyl) sodium 4,4′-dichloro-2,2′,6,6′-biphenyltetrasulfonate obtained in Example 3, 0.91 g of SUMIKAEXCEL (manufactured by Sumitomo Chemical Company, registered trademark) PES 3600P (Mn=2.7×10⁴, Mw=4.5×10⁴) having a structure represented by the following formula:

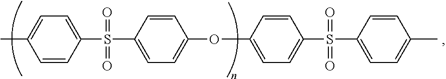

1.16 g of 2,2′-bipyridine, and 24 g of DMSO was raised up to 60° C., and 1.95 g of nickel (0) bis(cyclooctadiene) was added thereto, followed by stirring for 5 hours. The obtained reaction mixture was poured into 100 g of a 25% aqueous nitric acid solution, the precipitate was filtered, and a cake obtained by the filtration was washed three times with water. Anhydrous lithium bromide (1.41 g) and 18 g of N-methyl-2-pyrrolidone were added to the washed cake, and the obtained mixture was stirred at 120° C. for 24 hours.

The obtained mixture was poured into 200 g of 19% hydrochloric acid, and crystals were precipitated, followed by filtration. The obtained cake was washed with water and dried, thereby obtaining 0.88 g of a polymer having a structural unit originating from 4,4′-dichloro-2,2′,6,6′-biphenyltetrasulfonic acid shown below. Mw of the obtained polymer was 7.4×10⁴ and Mn thereof was 4.5×10⁴. The obtained polymer was water-insoluble.

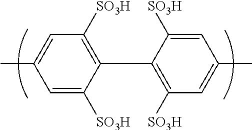

Preparation of Polymer Electrolyte Membrane

The obtained polymer (0.8 g) was dissolved in 7.2 g of DMSO, thereby preparing a polymer solution. Thereafter, the obtained polymer solution was cast onto a glass substrate, followed by drying at 80° C. for 2 hours under normal pressure, thereby removing the solvent. Subsequently, the resultant was treated with 6% hydrochloric acid and washed with ion exchange water, thereby forming a polymer electrolyte membrane having a thickness of about 30 μm. The ion exchange capacity of the obtained polymer electrolyte membrane was 1.7 meq/g, and the weight fraction of the structural unit having a sulfonic acid group in the polymer was calculated to be 0.19. In addition, the water vapor permeability coefficient of the obtained polymer electrolyte membrane was 5.1×10⁻¹⁰ mol/sec/cm.

### Example 7

### Polymer Synthesis

The temperature of a solution which contained 1.05 g (1.35 mmol) of tris(2,2-dimethyl-1-propyl) sodium 4,4′-dichloro-2,2′,6,6′-biphenyltetrasulfonate obtained in Example 3, 0.71 g of SUMIKAEXCEL (manufactured by Sumitomo Chemical Company, registered trademark) PES 3600P (Mn=2.7×10⁴, Mw=4.5×10⁴) having a structure represented by the following formula:

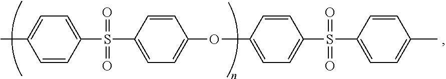

1.15 g of 2,2′-bipyridine, and 24 g of NMP was raised up to 60° C., and 1.93 g of nickel (0) bis(cyclooctadiene) was added thereto, followed by stirring for 5 hours. The obtained reaction mixture was poured into 100 g of a 25% aqueous nitric acid solution, the precipitate was filtered, and a cake obtained by the filtration was washed three times with water. Anhydrous lithium bromide (1.41 g) and 23 g of N-methyl-2-pyrrolidone were added to the washed cake, and the obtained mixture was stirred at 120° C. for 24 hours.

The obtained mixture was poured into 200 g of 19% hydrochloric acid, and crystals were precipitated, followed by filtration. The obtained cake was washed with water and dried, thereby obtaining 0.79 g of a polymer having a structural unit originating from 4,4′-dichloro-2,2′,6,6′-biphenyltetrasulfonic acid shown below. Mw of the obtained polymer was 6.6×10⁴ and Mn thereof was 4.5×10⁴. The obtained polymer was water-insoluble.

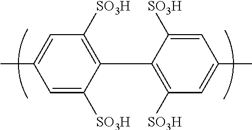

Preparation of Polymer Electrolyte Membrane

The obtained polymer (0.6 g) was dissolved in 5.4 g of DMSO, thereby preparing a polymer solution. Thereafter, the obtained polymer solution was cast onto a glass substrate, followed by drying at 80° C. for 2 hours under normal pressure, thereby removing the solvent. Subsequently, the resultant was treated with 6% hydrochloric acid and washed with ion exchange water, thereby forming a polymer electrolyte membrane having a thickness of about 45 p.m. The ion exchange capacity of the obtained polymer electrolyte membrane was 2.0 meq/g, and the weight fraction of the structural unit having a sulfonic acid group in the polymer was calculated to be 0.24. In addition, the water vapor permeability coefficient of the obtained polymer electrolyte membrane was 8.7×10⁻¹⁰ mol/sec/cm.

### Example 8

### Polymer Synthesis

To a flask provided with an azeotropic distillation device, 10.2 g (54.7 mmol) of 4,4′-dihydroxy-1,1′-biphenyl, 8.32 g (60.2 mmol) of potassium carbonate, 96 g of DMAc, and 50 g of toluene were introduced in a nitrogen atmosphere. Toluene was heated to reflux at a bath temperature of 155° C. for 2.5 hours to cause azeotropic dehydration of the moisture in the system. After the generated water and toluene were distilled away, the residue was cooled to room temperature, and 22.0 g (76.6 mmol) of 4,4′-dichlorodiphenylsulfone was added thereto. The temperature of the obtained mixture was raised up to 160° C., followed by stirring for 14 hours while keeping this temperature. After cooled, the reaction solution was added to a mixed solution containing 1000 g of methanol and 200 g of 35% hydrochloric acid, and the precipitated sediment was filtered. Thereafter, the resultant was washed with ion exchange water until the washings became neutral, followed by drying. 27.2 g of the obtained crude product was dissolved in 97 g of DMAc, and the insoluble matter was removed by filtration. Subsequently, the filtrate was added to a mixed solution containing 1100 g of methanol and 100 g of 35% by weight hydrochloric acid, the precipitated sediment was filtered, and the resultant was washed with ion exchange water until the washings became neutral, followed by drying. In this manner, 25.9 g of aromatic polyether A represented by the following formula was obtained. Mw of the obtained aromatic polyether A was 3.2×10³, and Mn thereof was 1.7×10³.

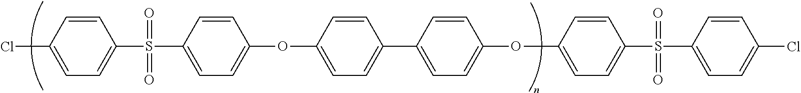

wherein n represents the number of a repeating unit.

The temperature of a solution which contained 0.90 g (1.16 mmol) of tris(2,2-dimethyl-1-propyl) sodium 4,4′-dichloro-2,2′,6,6′-biphenyltetrasulfonate obtained in Example 3, 0.38 g of the aromatic polyether A, 2.53 g of 2,2′-bipyridine, and 8 g of NMP was raised up to 60° C., and 4.24 g of nickel (0) bis(cyclooctadiene) was added thereto, followed by stirring for 5 hours. The obtained reaction mixture was poured into 100 g of a 25% aqueous nitric acid solution, the precipitate was filtered, and a cake obtained by the filtration was washed three times with water. Anhydrous lithium bromide (1.01 g) and 11 g of NMP were added to the washed cake, and the obtained mixture was stirred at 120° C. for 24 hours. The obtained mixture was poured into 200 g of 19% hydrochloric acid, and crystals were precipitated, followed by filtration. The obtained cake was washed with water and dried, thereby obtaining 0.63 g of a polymer having a structural unit originating from 4,4′-dichloro-2,2′,6,6′-biphenyltetrasulfonic acid shown below. Mw of the obtained polymer was 3.6×10⁴ and Mn thereof was 1.8×10⁴. The obtained polymer was water-insoluble.

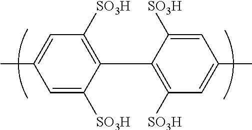

Preparation of Polymer Electrolyte Membrane

The obtained polymer (0.6 g) was dissolved in 3.4 g of DMSO, thereby preparing a polymer solution. Thereafter, the obtained polymer solution was cast onto a PET film, followed by drying at 80° C. for 2 hours under normal pressure, thereby removing the solvent. Subsequently, the resultant was treated with 6% hydrochloric acid and washed with ion exchange water, thereby forming a polymer electrolyte membrane having a thickness of about 30 μm. The ion exchange capacity of the obtained polymer electrolyte membrane was 4.2 meq/g, and the weight fraction of the structural unit having a sulfonic acid group in the polymer was calculated to be 0.49. In addition, the water vapor permeability coefficient of the obtained polymer electrolyte membrane was 4.1×10⁹ mol/sec/cm.

Values obtained by dividing the water vapor permeability coefficient of the polymer electrolyte membrane of the above examples by the weight fraction of the structural unit having a sulfonic acid group relative to the polymer constituting the polymer electrolyte membrane are summarized in Table 1.

TABLE 1

| | (Water vapor permeability coefficient)/(Weight fraction |
|---|---|
| | of structural unit having sulfonic acid group |
| | relative to the polymer) [mol/sec/cm] |
| Example 6 | 2.6 × 10⁻⁹ |
| Example 7 | 3.6 × 10⁻⁹ |
| Example 8 | 8.2 × 10⁻⁹ |

### Example 9

### Synthesis of Tetrakis(2,2-dimethyl-1-propyl) 4,4′-dichloro-2,2′,6,6′-biphenyltetrasulfonate

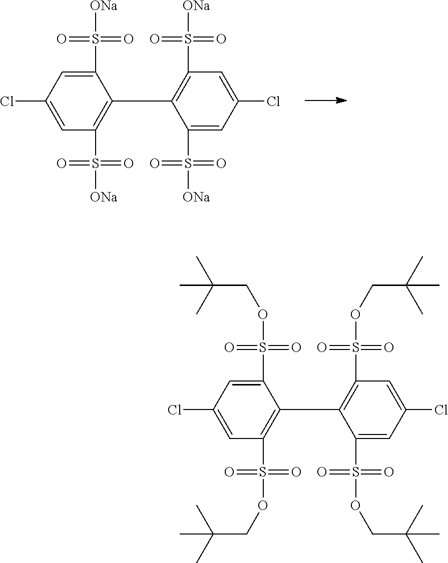

Chloroform (1.0 g) and 0.33 g of phosphorus pentachloride were added to 0.05 g of the tetrasodium 4,4′-dichloro-2,2′,6,6′-biphenyltetrasulfonate synthesized in Example 2. The temperature of the obtained mixture was raised up to 60° C. and kept at this temperature for 6 hours, and the reaction mixture was poured into 10.0 g of water. After liquid separation, the organic phase was concentrated and dried. The obtained concentrated residue was called a “concentrate 1”. Meanwhile, a 1.65 M hexane solution of n-butyllithium (0.4 mL, 0.65 mmol) was added dropwise to a solution including 0.07 g of 2,2-dimethyl-1-propanol and 1.0 g of anhydrous tetrahydrofuran, at 25° C., and the resultant was kept at this temperature for 30 minutes. The “concentrate 1” was incorporated to this solution, and the resultant was kept at 25° C. for 14 hours. The reaction mixture was purified with a silica gel plate (PLC Silica gel 60 RP-18 F₂₅₄ₛ, mobile phase; acetonitrile), and the obtained eluate was concentrated and dried, thereby obtaining 0.03 g (yield 45%) of white solid tetrakis(2,2-dimethyl-1-propyl) 4,4′-dichloro-2,2′,6,6′-biphenyltetrasulfonate.

¹H-NMR (heavy chloroform, δ (ppm)): 0.88 (s, 36H), 3.83 (s, 8H), 8.12 (s, 4H)

### INDUSTRIAL APPLICABILITY

According to the present invention, it is possible to provide a monomer that can impart ion conductivity to a macromolecule having an elimination group, a novel polymer that is obtained by polymerizing the monomer, a novel polymer electrolyte that contains the polymer, and the like.

1. A biphenyltetrasulfonic acid compound represented by the formula (1): 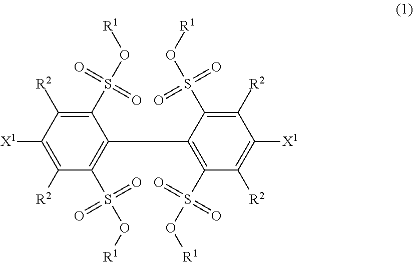
wherein R¹ each independently represents a hydrogen atom, a cation, or a hydrocarbon group having 1 to 20 carbon atoms that may have a substituent; R² each independently represents a hydrogen atom, an alkyl group having 1 to 20 carbon atoms that may have a substituent, an alkoxy group having 1 to 20 carbon atoms that may have a substituent, an aryl group having 6 to 20 carbon atoms that may have a substituent, an aryloxy group having 6 to 20 carbon atoms that may have a substituent, an aralkyl group having 7 to 20 carbon atoms that may have a substituent, or an aralkyloxy group having 7 to 20 carbon atoms that may have a substituent; and X¹ each independently represents a chlorine atom, a bromine atom, or an iodine atom.

2. The biphenyltetrasulfonic acid compound according to claim 1,
wherein in the formula (1), at least one R¹ is a hydrogen atom or a cation, and at least one R² is a hydrogen atom.

3. The biphenyltetrasulfonic acid compound according to claim 1,
wherein in the formula (1), at least one R¹ is an alkyl group having 1 to 6 carbon atoms.

4. A method for producing a biphenyltetrasulfonic acid compound represented by the formula (1): 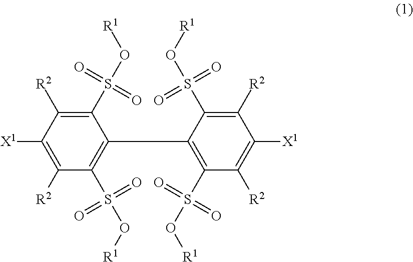
wherein R¹ each independently represents a hydrogen atom, a cation, or a hydrocarbon group having 1 to 20 carbon atoms that may have a substituent; R² each independently represents a hydrogen atom, an alkyl group having 1 to 20 carbon atoms that may have a substituent, an alkoxy group having 1 to 20 carbon atoms that may have a substituent, an aryl group having 6 to 20 carbon atoms that may have a substituent, an aryloxy group having 6 to 20 carbon atoms that may have a substituent, an aralkyl group having 7 to 20 carbon atoms that may have a substituent, or an aralkyloxy group having 7 to 20 carbon atoms that may have a substituent; X1 represents a chlorine atom, a bromine atom, or an iodine atom; and X2 represents a chlorine atom, a bromine atom, or an iodine atom, the method comprising:
a coupling reaction step of causing a coupling reaction of a benzenedisulfonic acid compound represented by the formula (2): 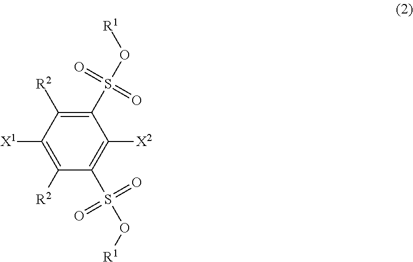
wherein R1, R2, and X1 have the same definitions as those described above.

5. The production method according to claim 4,
wherein the coupling reaction step is a step of causing a coupling reaction of the benzenedisulfonic acid compound represented by the formula (2) in the presence of metallic copper and monovalent copper halide.

6. A method for producing a benzenedisulfonic acid compound represented by the formula (2): 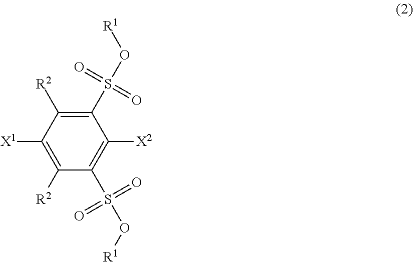
wherein R¹ each independently represents a hydrogen atom, a cation, or a hydrocarbon group having 1 to 20 carbon atoms that may have a substituent; R² each independently represents a hydrogen atom, an alkyl group having 1 to 20 carbon atoms that may have a substituent, an alkoxy group having 1 to 20 carbon atoms that may have a substituent, an aryl group having 6 to 20 carbon atoms that may have a substituent, an aryloxy group having 6 to 20 carbon atoms that may have a substituent, an aralkyl group having 7 to 20 carbon atoms that may have a substituent, or an aralkyloxy group having 7 to 20 carbon atoms that may have a substituent; X1 represents a chlorine atom, a bromine atom, or an iodine atom; and X2 represents a chlorine atom, a bromine atom, or an iodine atom, the method comprising:
a step of generating a diazonium compound by reacting an aniline compound represented by the formula (3): 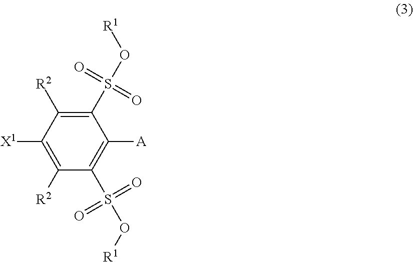
wherein R1, R2, and X1 have the same definitions as those described above; and A represents NH2, with a nitrous acid compound; and
a step of obtaining the benzenedisulfonic acid compound represented by the formula (2) by reacting the diazonium compound obtained in the above step with a halogen compound.

7. A polymer comprising a structural unit originating from the biphenyltetrasulfonic acid compound according to claim 1.

8. The polymer according to claim 7 further comprising a structural unit represented by the formula (X):
Ar⁰ (X)
wherein Ar0 represents an aromatic group which may have a substituent.

9. The polymer according to claim 7 further comprising a structural unit represented by the formula (5):
(Ar¹—Y¹)ₐ—Ar²—Z¹(Ar³—Y²)_{b}—Ar⁴—Z²]_{c}—]_{ₙ}Ar¹−Y¹ₐAr²]— (5
wherein a, b, and c each independently represents 0 or 1; n represents an integer of 2 or more; Ar1, Ar2, Ar3, and Ar4 each independently represents an aromatic group which may have a substituent; Y1 and Y2 each independently represents a single bond, a carbonyl group, a sulfonyl group, an isopropylidene group, a hexafluoroisopropylidene group, or a fluorene-9,9-diyl group; and Z1 and Z2 each independently represents an oxygen atom or a sulfur atom.

10. The polymer according to claim 7 further comprising a structural unit represented by the formula (5′):
(Ar¹—Y¹)ₐ—Ar²—Z¹(Ar³—Y²)_{b}—Ar⁴—Z²]_{c}—]_{_{n′}}Ar¹−Y¹ₐAr²]— (5′
wherein a, b, and c each independently represents 0 or 1; n′ represents an integer of 5 or more; Ar1, Ar2, Ar3, and Ar4 each independently represents an aromatic group which may have a substituent; Y1 and Y2 each independently represents a single bond, a carbonyl group, a sulfonyl group, an isopropylidene group, a hexafluoroisopropylidene group, or a fluorene-9,9-diyl group; and Z1 and Z2 each independently represents an oxygen atom or a sulfur atom.

11. (canceled)

12. A method for producing a polymer comprising a step of polymerizing a composition that contains a macromolecule including a structural unit represented by the formula (5):
(Ar¹—Y¹)ₐ—Ar²—Z¹(Ar³—Y²)_{b}—Ar⁴—Z²]_{c}—]_{ₙ}Ar¹−Y¹ₐAr²]— (5
wherein a, b, and c each independently represents 0 or 1; n represents an integer of 2 or more; Ar1, Ar2, Ar3, and Ar4 each independently represents an aromatic group which may have a substituent; Y1 and Y2 each independently represents a single bond, a carbonyl group, a sulfonyl group, an isopropylidene group, a hexafluoroisopropylidene group, or a fluorene-9,9-diyl group; and Z1 and Z2 each independently represents an oxygen atom or a sulfur atom, and a biphenyltetrasulfonic acid compound represented by the formula (1): 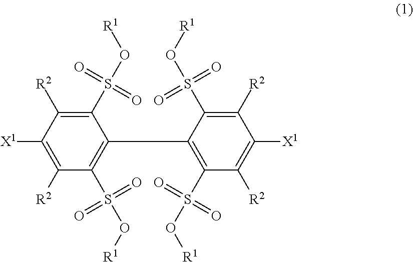
wherein R1 each independently represents a hydrogen atom, a cation, or a hydrocarbon group having 1 to 20 carbon atoms that may have a substituent; R2 each independently represents a hydrogen atom, an alkyl group having 1 to 20 carbon atoms that may have a substituent, an alkoxy group having 1 to 20 carbon atoms that may have a substituent, an aryl group having 6 to 20 carbon atoms that may have a substituent, an aryloxy group having 6 to 20 carbon atoms that may have a substituent, an aralkyl group having 7 to 20 carbon atoms that may have a substituent, or an aralkyloxy group having 7 to 20 carbon atoms that may have a substituent; and X1 each independently represents a chlorine atom, a bromine atom, or an iodine atom, in the presence of a nickel compound.

13. A method for producing a polymer comprising:
a step of polymerizing a composition that contains a macromolecule including a structural unit represented by the formula (5′):
(Ar¹—Y¹)ₐ—Ar²—Z¹(Ar³—Y²)_{b}—Ar⁴—Z²]_{c}—]_{_{n′}}Ar¹−Y¹ₐAr²]— (5′
wherein a, b, and c each independently represents 0 or 1; n′ represents an integer of 5 or more; Ar1, Ar2, Ar3, and Ar4 each independently represents an aromatic group which may have a substituent; Y1 and Y2 each independently represents a single bond, a carbonyl group, a sulfonyl group, an isopropylidene group, a hexafluoroisopropylidene group, or a fluorene-9,9-diyl group; and Z1 and Z2 each independently represents an oxygen atom or a sulfur atom, and a biphenyltetrasulfonic acid compound represented by the formula (1): 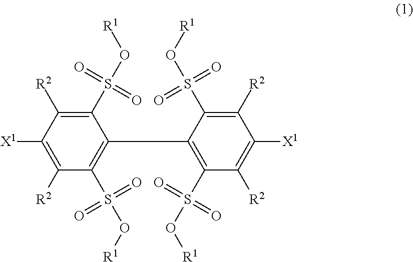
wherein R1 each independently represents a hydrogen atom, a cation, or a hydrocarbon group having 1 to 20 carbon atoms that may have a substituent; R2 each independently represents a hydrogen atom, an alkyl group having 1 to 20 carbon atoms that may have a substituent, an alkoxy group having 1 to 20 carbon atoms that may have a substituent, an aryl group having 6 to 20 carbon atoms that may have a substituent, an aryloxy group having 6 to 20 carbon atoms that may have a substituent, an aralkyl group having 7 to 20 carbon atoms that may have a substituent, or an aralkyloxy group having 7 to 20 carbon atoms that may have a substituent; and X1 each independently represents a chlorine atom, a bromine atom, or an iodine atom, in the presence of a nickel compound.

14. A polymer electrolyte comprising the polymer according to claim 7.

15. A polymer electrolyte membrane comprising the polymer electrolyte according to claim 14.

16. A polymer electrolyte composite membrane comprising the polymer electrolyte according to claim 14; and a porous substrate.

17. A catalyst composition comprising the polymer electrolyte according to claim 14; and a catalyst component.

18. A membrane electrode assembly comprising the polymer electrolyte membrane according to claim 15.

19. A polymer electrolyte fuel cell comprising the membrane electrode assembly according to claim 18.

20. A membrane electrode assembly comprising the polymer electrolyte composite membrane according to claim 16.

21. A membrane electrode assembly comprising the catalyst composition according to claim 17.
